(19) United States
(12) Patent Application Publication
(10) Pub. No.: US 2025/0375521 A1
(43) Pub. Date: Dec. 11, 2025

(54) Methods of Genetically Modifying Cells for Altered Codon-Anti-Codon Interactions

(71) Applicant: The Board of Trustees of the Leland Stanford Junior University, Stanford, CA (US)

(72) Inventors: Ansuman Satpathy, Redwood City, CA (US); Caleb Lareau, Redwood City, CA (US)

(21) Appl. No.: 18/876,574

(22) PCT Filed: Jun. 20, 2023
(86) PCT No.: PCT/US2023/025711

§ 371 Date: Dec. 18, 2024

Related U.S. Application Data
(60) Provisional application No. 63/353,715, filed on Jun. 20, 2022.

(51) Int. Cl. A61K 40/11; A61K 35/17; A61K 40/15; A61K 40/31; A61K 40/32; A61K 40/50; C07K 14/725; C12N 5/0783; C12N 5/10; C12N 9/00; C12Q 1/68

(52) U.S. Cl. CPC A61K 40/11; A61K 35/17; A61K 40/15; A61K 40/31; A61K 40/32; A61K 40/50; C07K 14/7051; C12N 5/0637; C12N 5/0646; C12N 5/10; C12N 9/00; C12Q 1/68; C12N 2510/00; C12Q 2600/156

(57) ABSTRACT

Provided are methods of genetically modifying cells. In certain embodiments, the methods comprise modifying a coding region of a mitochondrial gene of the cell. According to some embodiments, the modification results in increased translation of a messenger RNA (mRNA) encoded by the mitochondrial gene by increasing the affinity of a codon-anti-codon interaction during translation of the mRNA as compared to the affinity of the codon-anti-codon interaction prior to the modifying. In certain embodiments, the modification results in decreased translation of an mRNA encoded by the mitochondrial gene by decreasing the affinity of a codon-anti-codon interaction during translation of the mRNA as compared to the affinity of the codon-anti-codon interaction prior to the modifying. Also provided are populations of the genetically modified cells, compositions comprising such populations, and methods of administering the compositions to a subject as a cell-based therapy.

### CROSS-REFERENCE TO RELATED APPLICATIONS

This application claims the benefit of U.S. Provisional Patent Application No. 63/353,715, filed Jun. 20, 2022, which application is incorporated herein by reference in its entirety.

### INTRODUCTION

Translation elongation is a major determinant of the composition of the proteome, affecting the amounts of each protein, the errors within each protein, and protein folding. A codon is a sequence of three nucleotides in messenger RNA (mRNA) that are read simultaneously by the anticodon sequence of tRNA within a ribosome during translation. During translation elongation, each triplet nucleotide codon in mRNA is decoded in the A-site of the ribosome by interactions with the anticodon of its cognate tRNA (aminoacyl or charged tRNA), resulting in insertion of an amino acid, followed by a precise three base translocation of the mRNA (and tRNA) to maintain the reading frame.

In the standard genetic code, of the 64 triplets or codons, 61 codons correspond to the 20 amino acids. While Met and Trp are encoded by one codon each, the other 18 amino acids are encoded by two to six different codons, sometimes referred to as codon degeneracy. Different codons that encode the same amino acid are known as synonymous codons. Even though synonymous codons encode the same amino acid, the distribution of these codons in a genome is not random. Certain synonymous codons are preferred over other synonymous codons, leading to different frequencies of occurrence of synonymous codons within a genome, sometimes referred to as codon usage bias.

The efficiency of decoding of different synonymous codons by anticodons might not be the same by virtue of being different in their nucleotide sequences. Apart from this, the association rate of ternary complex formation between an anticodon, the A-site of ribosome, and the mRNA may be dissimilar for different synonymous codons. In addition, the impact of codon context during translation and the effect of certain sequences in mRNA on ribosome movement during translation are attributes of synonymous codons. Therefore, synonymous codons can influence gene expression at both the posttranscriptional and translational levels. Genome-wide analyses have determined that specific codons and codon combinations modulate ribosome speed and facilitate protein folding. In addition to tRNA availability, interactions between adjacent codons and wobble base pairing also determine the rate and efficiency of translation.

Translation in humans takes place in the cytosol and mitochondria. Mitochondrial translation is responsible for the maintenance of the cellular energetic balance through synthesis of proteins involved in oxidative phosphorylation. This is required for adenosine triphosphate (ATP) production and the folding of the cristae. Therefore, impaired mitochondrial translation results in severe combined respiratory chain dysfunction leading to diminished ATP production and consequent cellular energy deficit.

### SUMMARY

Provided are methods of genetically modifying cells. In certain embodiments, the methods comprise modifying a coding region of a mitochondrial gene of the cell. According to some embodiments, the modification results in increased translation of a messenger RNA (mRNA) encoded by the mitochondrial gene by increasing the affinity of a codon-anti-codon interaction during translation of the mRNA as compared to the affinity of the codon-anti-codon interaction prior to the modifying. In certain embodiments, the modification results in decreased translation of an mRNA encoded by the mitochondrial gene by decreasing the affinity of a codon-anti-codon interaction during translation of the mRNA as compared to the affinity of the codon-anti-codon interaction prior to the modifying. Also provided are populations of the genetically modified cells, compositions comprising such populations, and methods of administering the compositions to a subject as a cell-based therapy.

### BRIEF DESCRIPTION OF THE FIGURES

FIG. 1a-1g: Identification of a mosaic synonymous mtDNA variant with a CD8⁺ T-cell restricted selection bias. (a) Schematic of longitudinal peripheral blood samples obtained from a healthy donor. A total of 5 draws spanning 150 days were taken and processed with the mtscATAC-seq assay. (b) Summary of somatic mtDNA mutations called from aggregated draws. The overall median heteroplasmy is noted, as well as the m.7076A>G allele that is present at a 47.3% pseudobulk heteroplasmy. (c) Distribution of single-cell heteroplasmy across all cells profiled for the m.7076A>G allele. (d) Schematic of the mitochondrial genome with genes contributing to indicated complexes of the respiratory chain (I, III, IV, and V) being color-coded. The asterisk under the MT-CO1 genes denotes the position of the m.7076 allele. Annotation of the mutation, including the protein consequence (p.Gly391=) of the synonymous variant is shown below. (e) Uniform manifold approximation and projection (UMAP) of accessible chromatin profiles of PBMCs assayed via mtscATAC-seq colored by the density of the m.7076A (wildtype) allele. (f) Cluster annotation and cell type labeling of the same cells as in (e). The arrow indicates the CD8⁺ T effector memory (CD8⁺ TEM) population. (g) Ratio of homoplasmic cells with wildtype m.7076A to mutant m.7076G variants across indicated cell state clusters. The arrow highlights the CD8⁺ TEM cell state as the population with the greatest skew (p<2.2e-16; binomial test).

FIG. 2a-2d: Stable expression of MT-CO1 transcript but altered clone size of CD8⁺ effector memory T cells carrying the mutant m.7076G allele. (a) Heteroplasmy of m.7076A>G in indicated T cell subpopulations based on scRNA-seq. The size of each dot is scaled by the abundance of cells in each cell state. (b) Comparison of MT-CO1 expression across indicated cell states stratified by the m.7076A and G alleles. P-values from a Wilcox test comparing log₂ MT-CO1 UMI counts per cell were not significant at type I error of 0.05. (c) Comparison of TCR done sizes between CD4⁺ and CD8⁺ T cells with homoplasmic m.7076A or m.7076G alleles. P-values are shown for a Wilcox test comparing clone sizes which were significant at type I error of 0.05. (d) Differential gene expression of all genes between cells with homoplasmic wildtype m.7076A vs. mutant m.7076G within the indicated CD8⁺ T cel compartments. 0 genes were differentially expressed in naive CD8⁺ T cells whereas 32 were differentially expressed in CD8⁺ TEM cells, including the 5 highlighted in the text. No other differentially expressed genes were observed in other T cell subsets.

FIG. 3a-3d: Mitochondrial ribosome profiling reveals translational stalling of the mutant m.7076A>G allele. (a) Summary overview of codon, anticodon, tRNA, and codon:anticodon recognition mechanisms for glycine in the human nuclear and mitochondrial genomes. (b) Polysome profile following sucrose gradient and western blots of isolated fractions for mitochondrial ribosome profiling. MRLP11 and RPS6 were blotted to identify enrichment of mitochondrial and cytoplasmic ribosomes, respectively (c) Summary of heteroplasmy from ribosome profiling libraries (fractions 5-9, see panel (b)) showing a relative increase of the mutant m.7076A>G allele in ribosomal bound fractions versus input RNA. Statistical significance was determined using a Fisher's exact test of 7076A and G alleles summed between replicates. (d) Schematic of the functional effect of the synonymous m.7076A>G variant. Due to decreased codon:anticodon affinity of the m.7076A>G allele, there is an increase in stalling of the MT-CO1 transcript, prohibiting effective translation.

FIG. 4a-4e: A new ontogeny of synonymous mtDNA mutations reveals patterns of human germline variation mediated by wobble-dependent translation. (a) Classification of all 8,284 possible synonymous mtDNA variants based on Watson-Crick-Franklin (WCF) or wobble-dependent base-pairing at either the reference or alternative allele. (b) The abundance of each synonymous variant class at different thresholds for the number of carriers for two population-scale sequencing databases. The difference in the percent of all synonymous variants is plotted for the four variant classes (null: dotted line at 0). (c) The proportion of the amino acid encoded by the codons relevant for the Wobble→WCF variants. The observed variants represent Wobble→WCF present in at least 100 Individuals from HelixMTdb. The test statistic is a Chi-squared test for the number of observed Wobble→WCF variants encoding each amino acid. (d) Comparison of % observed (right) versus expected (left) for 87 haplogroup-defining, synonymous mtDNA mutations. P-values represent the statistical significance of a two-sided binomial test statistic. (e) Inter-species conservation at wobble-position nucleotides in mitochondrial codons. Reference alleles that can be mutated to different outcomes are specified and grouped with the number of wobble-position codons in each class noted. P-values represent a Wilcoxon test.

FIG. 5a-5d: Complex human phenotypes are associated with synonymous mtDNA mutations. (a) Reanalysis of statistically significant associations in protein-coding genes between mtDNA variants measured by a genotyping array and complex traits assayed in the UKBB²⁹. (b) Correlation of effect sizes for common variants estimated from WGS (y-axis; computed in this study) and prior array-based work²⁹. Only variant-quantitative trait pairs that were statistically significant in the array-based study²⁹ are noted, and the Pearson correlation between the effect sizes is shown. (c) Manhattan plots of all synonymous mtDNA variants stratified by wobble-dependent ontogeny using a large biobank of whole-genome sequencing data. (d) Same as in (c) but for binary traits. Each dot represents a unique trait-genotype association with a p-value <1×10⁻⁴.

FIG. 6a-6e: Wobble-dependent translation reveals patterns of synonymous somatic mtDNA variation in tumors and healthy tissues. (a) Comparison of observed (right) versus expected (left) somatic synonymous mtDNA mutations based on three tumor atlas sequencing cohorts (TCGA, IMPACT, PCAWG). P-values represent the statistical significance of a two-sided binomial test statistic. (b) Stratification of synonymous variants by tRNA base in the anticodon wobble position. Faded colors represent the expected numbers of mutations under a null model. Percents represent the fraction of mutations that are WCF→Wobble. Chi-squared test of association between observed and expected mutation values based on numbers of potential or observed mutations in each synonymous mutation class are shown below each pair of bar plots. (c) Gene-level abundances of missense and WCF→Wobble variants comparing expected (based on all possible variants) versus observed variants across the entire dataset. The Pearson correlation and p-value from the Pearson correlation test are noted. (d) Same as in (a) but for somatic mutations identified in the mtDNA genome in one or more non-cancerous GTEx tissues¹³. (e) The proportion of synonymous variants that are Wobble→WCF within individual tissues. 22% (noted by the asterisk) is the abundance under the null. Tissues are rank-ordered by statistical significance using the p-value from a two-sided binomial test.

FIG. 7a-7d: Further information for selection against mutant m.7076G in CD8⁺ TEM. (a) Heatmap of single-cells comparing the coverage of both m.7076 alleles against heteroplasmy. (b) Compare to FIG. 1e with the smoothed representation. (c) Histograms comparing the 16 most common cell types from the Azimuth/Bridge Integration annotation. The red arrow highlights the significant reduction of cells with the m.7076A>G variant specifically in CD8⁺ TEM cells, but not other cell types. (d) Longitudinal heteroplasmy of different cell populations over >150 days of sampling. The right panel is a zoom of the region on the left panel (note axis). Pseudobulk heteroplasmy estimates including the standard error of the mean are shown.

FIG. 8a-8d: Further information for selection against mutant m.7076A>G using scRNA-seq. (a) Overlap of TCR clones with respective m.7076 alleles. Each dot is a TCR clone (min. 2 cells) summarizing the number of cells with either wildtype m.7076A or mutant m.7076G homoplasmy. (b Unsupervised clustering and dimensionality reduction of T cells stratified by m.7076A or m.7076G homoplasmy. Black boxes around clusters 5 (98.1%) and 7 (95.9%) represent specific cell states that are primarily restricted to cells harboring the m.7076A allele. (c) Annotation of two highly expanded TCR clones restricted to cells homoplasmic for wildtype m.7076A. Blue represents TCR clones (n≥2 cells) that were not highly expanded. (d) Heteroplasmy of the m.7076A>G allele in indicated T cell subpopulations based on scRNA-seq after excluding clones A and B from panel (c). The size of each dot is scaled by the abundance of cells in each cell state.

FIG. 9a-9g: In vitro activation of T cells refines cell states depleted of the mutant 7076G allele. (a) Schematic of experimental design. T cells were isolated from Donor 1, in vitro activated, and cultured for 9 days before profiling via flow cytometry and ASAP-seq. (b) UMAP of accessible chromatin profiles and projected ratio of CD8 over CD4 antibody-derived tags from day 9 cells profiled via ASAP-seq. Arrow indicates a population highly enriched for the m.7076A (wildtype) allele. (c) Same as (b) but colored by the density of the m.7076A (wildtype) allele. (d) UMAP embedding colored by KLRG1 (top) and IL7R (bottom) antibody tag density. (e) UMAP colored by selected gene activity scores for four indicated gene loci. (f) UMAP colored by indicated cell state cluster. (g) Ratio of wildtype m.7076A to mutant m.7076G cells within indicated cell states. P-value represents the statistical significance of a two-sided binomial test statistic.

FIG. 10a-10f: Further information for analysis of the mitochondrial tRNA pool and impact on translational efficiency via the wobble effect. (a) Codon bias table for the nuclear genome. Within each amino acid, the ratio of observed codon usage over a null model of equal codon use per amino acid and colored by the log₂ of this measure is shown. (b) Same as (a) but for the 13 polypeptides encoded in the mitochondrial genome. (c) Coverage near the m.7076A>G variant. Red bars indicate the mutated codon with the m.7076 allele (noted with an asterisk). The relative proportion of reads phased to either allele per library is indicated. The translation pause ratio, defined as the fraction of reads from ribosome profiling over the RNA-seq libraries, is noted in the top left corner. (d) Three additional replicate fractions of MitoRibo-seq from an independent experiment of Donor 1 cells. Statistical significance was determined using a Fisher's exact test of 7076A and G alleles summed between replicates. (e) Synonymous alleles with distinct homoplasmy between HEK293 and Hela cell lines from a previous study²³. Two variants called out had sufficient coverage (>2 counts per 10 k; cp10 k) for further analysis. (f) Comparison of MitoRibo-seq read abundances between cell lines for two variants highlighted in (e). Shown are two variants that differ between cell lines, including one that impacted wobble-dependent translation (m.12372G>A; Wobble→WCF; Increased stalling at wobble allele) or not (m.9540T>C; WCF→WCF). Statistical test: two-sided Wilcoxon test.

FIG. 11a-11b: Further information comparing mtDNA synonymous mutation patterns in gnomAD sequencing data. (a) Comparison of % observed (color) versus expected (grey) synonymous mtDNA mutations from gnomAD for homoplasmic variants (all observed homoplasmic variants from the population). (b) Same as in (a) but for variants present in ≥100 healthy individuals based on gnomAD analysis, showing enrichment of more optimal Wobble→WCF synonymous codons. P-values represent the statistical significance of a two-sided binomial test statistic.

FIG. 12: Further information comparing mtDNA synonymous mutation patterns in healthy human tissues. (a) Comparison of % observed (color) versus expected (grey) synonymous mtDNA mutations from a healthy 47-year-old individual profiled with mtscATAC-seq for somatic heteroplasmic variants in PBMCs.

FIG. 13a-13d: A new ontogeny of synonymous mtDNA mutations in the murine genome via wobble-dependent translation. (a) Classification of all 7,957 possible synonymous mtDNA variants based on Watson-Crick-Franklin (WCF) or wobble-dependent base-pairing at either the reference or alternative allele. (b) Inter-species conservation at wobble-position nucleotides in mitochondrial codons for the murine genome. Reference alleles that can be mutated to different outcomes are specified and grouped with the number of wobble-position codons in each class noted. P-values represent a Wilcoxon test. (c) Comparison of observed (right) versus expected (left) heteroplasmic synonymous mtDNA mutations occurring in the pol-γ mutant mice³⁶ and (d) in healthy aging mice³⁷. P-values represent the statistical significance of a two-sided binomial test statistic.

### DETAILED DESCRIPTION

Before the methods and compositions of the present disclosure are described in greater detail, it is to be understood that the methods and compositions are not limited to particular embodiments described, as such may, of course, vary. It is also to be understood that the terminology used herein is for the purpose of describing particular embodiments only, and is not intended to be limiting, since the scope of the methods and compositions will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions. The upper and lower limits of these smaller ranges may independently be included in the smaller ranges and are also encompassed within the methods and compositions, subject to any specifically excluded limit in the stated range. Where the stated range includes one or both of the limits, ranges excluding either or both of those included limits are also included in the methods and compositions.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and compositions belong. Although any methods and compositions similar or equivalent to those described herein can also be used in the practice or testing of the methods and compositions, representative illustrative methods and compositions are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and compositions are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and compositions, which are, for clarity, described in the context of separate embodiments, may also be provided in combination in a single embodiment. Conversely, various features of the methods and compositions, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and compositions and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods of Genetically Modifying Cells

The present disclosure provides methods of genetically modifying cells. In certain aspects, the methods comprise modifying a coding region of a mitochondrial gene of a cell. According to some embodiments, the modification results in increased translation of a messenger RNA (mRNA) encoded by the mitochondrial gene by increasing the affinity of a codon-anti-codon interaction during translation of the mRNA as compared to the affinity of the codon-anti-codon interaction prior to the modifying. In certain embodiments, the modification results in decreased translation of an mRNA encoded by the mitochondrial gene by decreasing the affinity of a codon-anti-codon interaction during translation of the mRNA as compared to the affinity of the codon-anti-codon interaction prior to the modifying.

The methods of the present disclosure are based in part on the Inventors' unexpected identification of a first synonymous mitochondrial DNA (mtDNA) variant which results in a wobble-dependent interaction and is strongly selected against during T cell expansion, and a second synonymous mtDNA variant which increases codon-anti-codon affinity and is positively correlated with T cell expansion. With the benefit of the present disclosure, therefore, it will be appreciated that altering (e.g., increasing) codon-anti-codon affinity via genetic modification of a coding region of a mitochondrial gene may be employed to increase the proliferative capacity of cells (e.g., immune cells such as T cells, NK cells, or the like) and/or altered cell phenotype (e.g. memory, cytotoxic), where such increased proliferative capacity and/or altered cell phenotype is advantageous in a variety of contexts including but not limited to cell-based therapies, e.g., CAR-T cell therapies, engineered T cell therapies (T cells that express engineered T cell receptors (TCRs)), and the like.

According to some embodiments, the modification results in increased translation of the mRNA. When the modification results in increased translation of the mRNA, in certain embodiments, the modifying converts the codon-anti-codon interaction from a wobble-dependent interaction to a non-wobble-dependent interaction.

In certain embodiments, the modification results in decreased translation of the mRNA. When the modification results in decreased translation of the mRNA, according to some embodiments, the modifying converts the codon-anti-codon interaction from a non-wobble-dependent interaction to a wobble-dependent interaction.

The wobble position of a codon refers to the third nucleotide in a codon. Binding of a codon in an mRNA to the cognate tRNA is much “looser” in the third position of the codon. The genetic code is redundant whereby several different codons code for the same amino acid. Often, this redundancy is specified in the third codon position such that several codons with the same first two nucleotides, but different third position nucleotides, code for the same amino acids. This permits several types of non-Watson-Crick-Franklin (non-WCF) base pairing to occur at the third codon position. The four main wobble base pairs are guanine-uracil (G-U), hypoxanthine-uracil (I-U), hypoxanthine-adenine (I-A), and hypoxanthine-cytosine (I-C). However, in mitochondria, only 22 of the possible 64 tRNAs are present in the genome.

As used herein, a “wobble-dependent interaction” is a codon-anti-codon interaction that does not follow Watson-Crick-Franklin (WCF) base pair rules at the wobble position of the codon. By “non-wobble-dependent interaction” is meant a codon-anti-codon interaction that follows Watson-Crick-Franklin (WCF) base pair rules at each position of the codon.

As summarized above, in some embodiments, the methods of genetically modifying cells of the present disclosure comprise modifying a coding region of a mitochondrial gene of a cell. Human mitochondrial DNA (mtDNA) is a double-stranded, circular molecule that encodes 14 proteins. The coding region may be within any mitochondrial gene of interest. In some embodiments, the protein encoded by the gene is an enzyme in the mitochondrial electron transport chain. For example, the methods may comprise modifying a coding region of MT-ND1, MT-ND2, MT-ND3, MT-ND4L, MT-ND4, MT-ND5, MT-ND6, MT-CYB, MT-CO1, MT-CO2, MT-CO3, MT-ATP6, MT-ATP8, or any combination thereof. According to some embodiments, the methods comprise modifying a coding region of MT-CO1 (also referred to as mitochondrially encoded cytochrome C oxidase 1). In some embodiments, the methods comprise modifying a coding region of MT-RNR2 (humanin).

The sequences of mitochondrial protein-coding genes are known and can be found, e.g., in the mtDB—Human Mitochondrial Genome Database (www.mtdb.igp.uu.se) (see Ingman & Gylensten mtDB: Human Mitochondrial Genome Database, a resource for population genetics and medical sciences. (2006) Nucleic Acids Res 34:D749-D751); GenBank (www.ncbi.nlm.nih.gov/genbank); UniProt (www.uniprot.org); and elsewhere.

Any of a variety of suitable approaches may be employed for the modification of a coding region of a mitochondrial gene of interest. One approach for mitochondrial genome editing employs the DddA-derived cytosine base editor (DdCBE) architecture comprising a pair of MTS-TALE arrays linked to one DddAtox (DddA) half either from the G1333 or G1397 split and a uracil glycosylase inhibitor (UGI), where one TALE monomer has DddAtox-N and the other has DddAtox-C. This programmable tool uses the MTS from SOD2 and the cytochrome c oxidase subunit 8A (COX8A). Further details regarding this approach may be found, e.g., in Mok et al. (2020) Nature 583(7817):631-637.

A further approach for mitochondrial genome editing is mitochondrial ARCUS (mitoARCUS). The ARCUS gene-editing tool is derived from the chloroplast homodimeric homing endonuclease I-CreI of Chlamydomonas reinhardtii, which belongs to the LAGLIDADG motif meganuclease family. Native I-CreI is a homodimeric enzyme that introduces DNA double-strand breaks (DSBs) by binding to a pseudopalindromic 22-bp double-stranded DNA sequence. ARCUS-engineered nucleases are monomeric, with novel sequence specificity being achieved thorough in silico design and directed evolution. mitoARCUS uses a mitochondrial targeting sequence (MTS) from the nuclear gene encoding COX8 of complex IV. Further details regarding this approach may be found, e.g., in U.S. Pat. No. 8,021,867B2 and U.S. Pat. No. 9,683,257.

Additional suitable approaches for mitochondrial genome editing include those that employ zinc finger nucleases, e.g., mtZFN, sc-mtZFN, and the like. For example, the dimeric mitochondrial zinc-finger nuclease (mtZFN) architecture, containing obligatory heterodimeric, ELD(−) and KKR(+), FokI nuclease domains may be employed. Also by way of example is mitochondrial single-chain ZFN (sc-mtZFN) combining two FokI domains in a single polypeptide chain. Mitochondrial targeting is facilitated by a 49-amino-acid MTS from subunit F1β of human mitochondrial ATP synthase. Further details regarding these approaches may be found, e.g., in Doyon et al. (2011) Nat. Methods 8:74-79.

Further suitable approaches for mitochondrial genome editing include those that employ transcription activator-like effector nucleases, e.g., mitoTALEN, mitoTev-TALE, mitoTALENickase, and the like. The dimeric mitochondrially targeted transcription activator-like effector (TALE) nuclease (mitoTALEN) contains obligatory heterodimeric, ELD(−) and KKR(+), FokI nuclease domains. This programmable nuclease uses the MTS from superoxide dismutase 2 (SOD2) and/or the COX8 plus subunit 9 of Neurospora crassa ATPase 9 (COX8-Sub9). For mitoTev-TALE, the TALE domain is attached through a flexible linker to the I-TevI nuclease just after the MTS instead of FokI in the C terminus. I-TevI requires a CNNNG site to create DSBs. mitoTev-TALE uses the MTS from COX8-Sub9. The dimeric mitochondrially targeted TALE nickase (mitoTALENickase) contains obligatory heterodimeric, ELD(−) and KKR(+), FokI nuclease domains. One of the domains is catalytically inactive owing to a D450A amino acid modification. This programmable nickase uses the MTS from SOD2.

Examples of suitable approaches for modifying a coding region of a mitochondrial gene of interest include, but are not limited to, those described in Silva-Pinheiro et al. (2022) Nat Commun. 13(1):750 (relating to mitochondrial base editing via adeno-associated viral delivery); Mok et al. (2020) Nature 583(7817):631-637 (relating to a bacterial cytidine deaminase toxin which enables CRISPR-free mitochondrial base editing); Yin et al. (2022) Front Physiol. 13:883459 (relating to mitochondrial genome editing by CRISPR); Silva-Pinheiro & Minczuk (2022) Nat Rev Genet. 23(4):199-214; Ral et al. (2018) Essays Biochem. 62(3):455-465; and Yang et al. (2021) Computational and Structural Biotechnology Journal 19:3319-3329, the disclosures of which (including the references cited therein) are incorporated herein by reference in their entireties for all purposes.

Aspects of the present disclosure further include methods of genetically modifying a cell, wherein translation of an mRNA encoded by a mitochondrial gene of the cell is wobble-dependent, and wherein such methods comprise introducing into mitochondria of the cell an expression construct from which a transfer RNA (tRNA) is transcribed. According to such methods, the anti-codon of the tRNA is selected such that translation of the mRNA encoded by the mitochondrial gene is no longer wobble-dependent. Such methods find use in a variety of contexts, including those in which a synonymous variant is present and results in wobble-dependent translation of a mitochondrial gene (e.g., a gene that encodes an enzyme in the mitochondrial electron transport chain, such as MT-CO1 or the like), and wherein the situation is remedied by the tRNA transcribed from the expression construct. With the benefit of the present disclosure, it will be appreciated that supplying such a tRNA that confers a WCF codon-anti-codon interaction finds use, e.g., to increase the proliferative capacity of cells (e.g., immune cells such as T cells, NK cells, or the like) and/or altered cell phenotype (e.g. memory, cytotoxic), where such increased proliferative capacity and/or altered cell phenotype is advantageous in a variety of contexts including but not limited to cell-based therapies, e.g., CAR-T cell therapies, engineered T cell therapies (T cells that express engineered T cell receptors (TCRs)), and the like. The sequences of tRNAs which may be selected for use in the methods of the present disclosure are known and include those found in the T-psi-C tRNA sequence database (see Sajek et al. (2019) Nucleic Adds Research 48(d1):D256-D260) and the GtRNAdb 2.0 tRNA sequence database (see Chan & Lowe (2016) Nucleic Acids Research 44(D1):D184-D189), the disclosures of which are incorporated herein by reference in their entireties for all purposes. Approaches for delivering an expression construct of interest to mitochondria of a cell are available and include, but are not limited to, the MITO-Porter approach described in Yamada et al. (2017) Biomaterials 136:56-66, the disclosure of which is incorporated herein in its entirety for all purposes.

Aspects of the present disclosure further include methods of genetically modifying a cell, the methods comprising introducing into mitochondria of the cel a first nucleic acid encoding a tRNA, and a second nucleic acid encoding a protein. According to such methods, translation of the protein encoded by the second nucleic acid is wobble-dependent in the absence of the tRNA and non-wobble-dependent in the presence of the tRNA. Such methods find use, e.g., when it is desirable to control the expression in the mitochondria of the protein encoded by the second nucleic acid. For example, according to some embodiments, transcription of the tRNA encoded by the first nucleic acid is inducible. In certain embodiments, transcription of the tRNA is induced upon activation of a signaling pathway of the cell. By way of example, when the cel is an immune cell (e.g., a T cel), the transcription of the tRNA may induced upon activation of the immune cell, e.g., by a cytokine, by binding of a receptor expressed on the surface of the immune cell to an antigen, or the like.

According to some embodiments, the first nucleic acid and the second nucleic acid are the same nucleic acid. In certain embodiments, the first nucleic acid and the second nucleic acid are separate nucleic acids. The first and second nucleic acids may be provided as one or two circular or linear polynucleotides (a polymer composed of naturally-occurring and/or non-naturally-occurring nucleotides) that encode the tRNA and protein operably linked to suitable promoters, e.g., a constitutive or inducible promoters. In some embodiments, expression of the tRNA and/or protein is under the control of one or more exogenous (including heterologous) regulatory elements, e.g., promoter, enhancer, etc., present in the nucleic acid (expression construct), and operably linked to the region encoding the tRNA and/or protein. In some embodiments, expression of the tRNA and/or protein may be controlled by one or more endogenous regulatory elements, e.g., promoter, enhancer, etc., at or near a mitochondrial genomic locus into which the expression construct is inserted.

The first and second nucleic acids (expression constructs, e.g., vectors) can be suitable for replication and integration into the mitochondrial genome of eukaryotic (e.g., human) cells. The first and second nucleic acids may contain functionally appropriately oriented transcription and translation terminators, initiation sequences, and promoters useful for regulation of the expression of the nucleic acid encoding the tRNA and/or protein. The first and second nucleic acids optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

In certain embodiments, upon delivery of the first and second nucleic acids to mitochondria of the cell, one or both of the first and second nucleic acids are episomal (e.g., extra-chromosomal), where by “episome” or “episomal” is meant a polynucleotide that replicates independently of the cell's mitochondrial DNA. A non-limiting example of an episome that may be employed in the present methods is a plasmid.

According to some embodiments, upon delivery of the first and second nucleic acids to mitochondria of the cell, one or both of the first and second nucleic acids integrate into the mitochondrial genome of the cell. In certain embodiments, one or both of the first and second nucleic acids are adapted for site-specific integration into the mitochondrial genome. Any suitable approach for site-specific integration may be employed. Functional integration of an expression construct may be achieved through various means, including through the use of integrating vectors, including viral and non-viral vectors. In some instances, a retroviral vector, e.g., a lentiviral vector, may be employed. In some instances, a non-retroviral integrating vector may be employed. An integrating vector may be contacted with the cells in a suitable transduction medium, at a suitable concentration (or multiplicity of infection), and for a suitable time for the vector to infect the target cells, facilitating functional integration of the expression construct. Non-limiting examples of useful viral vectors include retroviral vectors, lentiviral vectors, adenoviral (Ad) vectors, adeno-associated virus (AAV) vectors, hybrid Ad-AAV vector systems, and the like.

Strategies for site-specific integration that find use in the methods of the present disclosure include those that employ homologous recombination, nonhomologous end-joining (NHEJ), and/or the like. Such strategies may employ a non-naturally occurring or engineered nuclease, including, but not limited to, zinc-ringer nucleases (ZNFs), meganucleases, transcription activator-like effector nucleases (TALENs)), or a CRISPR-Cas system. Eukaryotic cells utilize two distinct DNA repair mechanisms in response to DNA double strand breaks (DSBs): Homologous recombination (HR) and nonhomologous end-joining (NHEJ). Mechanistically, HR is an error-free DNA repair mechanism because it requires a homologous template to repair the damaged DNA strand. Because of its homology-based mechanism, HR has been used as a tool to site-specifically engineer the genome. Gene targeting by HR requires the use of two homology arms that flank the transgene/target site of interest. HR efficiency can be increased by the introduction of DSBs at the target site using specific rare-cutting endonucleases. The discovery of this phenomenon prompted the development of methods to create site-specific DSBs in the genome of different species. Various chimeric enzymes have been designed for this purpose over the last decade, namely ZFNs, meganucleases, and TALENs. ZFNs are modular chimeric proteins that contain a ZF-based DNA binding domain (DBD) and a FokI nuclease domain. DBD is usually composed of three ZF domains, each with 3-base pair specificity; the FokI nuclease domain provides a DNA nicking activity, which is targeted by two flanking ZFNs. Owing to the modular nature of the DBD, any site in a genome could be targeted. TALENs are similar to ZFNs except that the DBD is derived from transcription activator-Ike effectors (TALEs). The TALE DBD is modular, and it is composed of 34-residue repeats, and its DNA specificity is determined by the number and order of repeats. Each repeat binds a single nucleotide in the target sequence through only two residues.

Any of the methods of genetically modifying cells of the present disclosure may be performed on any eukaryotic cel type of interest. In certain embodiments, a method of the present disclosure is performed on a yeast cell, an insect (e.g., drosophila) cell, an amphibian (e.g., frog, e.g., Xenopus) cel, a plant cell, etc. According to some embodiments, the method is performed on a mammalian cel. Mammalian cells of interest include human cels, rodent cells, and the like. According to some embodiments, the method is performed on a population of peripheral blood mononuclear cells (PBMCs). In certain embodiments, the method is performed on an immune cell. For example, the method may be performed on a T cell, a B cell, a natural killer (NK) cell, a macrophage, a monocyte, a neutrophil, a dendritic cell, a mast cell, a basophil, an eosinophil, or any combination thereof. When the immune cell is a T cell, the T cel may be a naive T cell (T_{N}), cytotoxic T cell (T_{CTL}), memory T cell (T_{MEM}), T memory stem cell (T_{SCM}), central memory T cell (T_{CM}), effector memory T cell (T_{EM}), tissue resident memory T cell (T_{RM}), effector T cell (T_{EFF}), regulatory T cell (T_{REG}), helper T cell, CD4+ T cell, CD8+ T cell, virus-specific T cell, alpha beta T cell (T_{αβ}), gamma delta T cell (T_{γδ}), or the like. In certain embodiments, a method of genetically modifying a cell of the present disclosure is performed on a CD8+ T cell. According to some embodiments, a method of genetically modifying a cell of the present disclosure is performed on a CD8+ T cell. In certain embodiments, a method of genetically modifying a cell of the present disclosure is performed on an NK cell.

According to some embodiments, a method of genetically modifying a cell of the present disclosure is performed on a stem cell, e.g., mammalian (e.g., human) stem cell. For example, the stem cell may be an embryonic stem (ES) cell, adult stem cell, hematopoietic stem cell (HSC), induced pluripotent stem cell (iPSC), mesenchymal stem cell (MSC), neural stem cell (NSC), or any combination thereof.

In certain embodiments, prior to, subsequent to, or concurrently with the genetically modifying of the cell, the cell is engineered (further genetically modified) to express a receptor (e.g., a recombinant receptor) on its surface. For example, the cell may be engineered to express a chimeric antigen receptor (CAR), a T cell receptor (TCR) such as a recombinant TCR, a chimeric cytokine receptor (CCR), a chimeric chemokine receptor, a synthetic notch receptor (synNotch), a Modular Extracellular Sensor Architecture (MESA) receptor, a Tango receptor, a ChaCha receptor, a generalized extracellular molecule sensor (GEMS) receptor, a growth factor receptor, a cytokine receptor, a chemokine receptor, a switch receptor, an adhesion molecule, an integrin, an inhibitory receptor, a stimulatory receptor, an immunoreceptor tyrosine-based activation motif (ITAM)-containing receptor, an immunoreceptor tyrosine-based inhibition motif (ITIM)-containing receptor, a hormone receptor, a receptor tyrosine kinase, an immune receptor such as CD28, CD80, ICOS, CTLA4, PD1, PD-L1, BTLA, HVEM, CD27, 4-1BB, 4-1BBL, OX40, OX40L, DR3, GITR, CD30, SLAM, CD2, 2B4, TIM1, TIM2, TIM3, TIGIT, CD226, CD160, LAG3, LAIR1, B7-1, B7-H1, and B7-H3, a type I cytokine receptor such as Interleukin-1 receptor, Interleukin-2 receptor, Interleukin-3 receptor, Interleukin-4 receptor, Interleukin-5 receptor, Interleukin-6 receptor, Interleukin-7 receptor, Interleukin-9 receptor, Interleukin-11 receptor, Interleukin-12 receptor, Interleukin-13 receptor, Interleukin-15 receptor, Interleukin-18 receptor, Interleukin-21 receptor, Interleukin-23 receptor, Interleukin-27 receptor, Erythropoietin receptor, GM-CSF receptor, G-CSF receptor, Growth hormone receptor, Prolactin receptor, Leptin receptor, Oncostatin M receptor, Leukemia inhibitory factor, a type II cytokine receptor such as interferon-alpha/beta receptor, interferon-gamma receptor, Interferon type III receptor, Interleukin-10 receptor, Interleukin-20 receptor, Interleukin-22 receptor, Interleukin-28 receptor, a receptor in the tumor necrosis factor receptor superfamily such as Tumor necrosis factor receptor 2 (1B), Tumor necrosis factor receptor 1, Lymphotoxin beta receptor, OX40, CD40, Fas receptor, Decoy receptor 3, CD27, CD30, 4-1BB, Decoy receptor 2, Decoy receptor 1, Death receptor 5, Death receptor 4, RANK, Osteoprotegerin, TWEAK receptor, TACI, BAFF receptor, Herpesvirus entry mediator, Nerve growth factor receptor, B-cell maturation antigen, Glucocorticoid-induced TNFR-related, TROY, Death receptor 6, Death receptor 3, Ectodysplasin A2 receptor, a chemokine receptor such as CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, XCR1, ACKR1, ACKR2, ACKR3, ACKR4, CCRL2, a receptor in the epidermal growth factor receptor (EGFR) family, a receptor in the fibroblast growth factor receptor (FGFR) family, a receptor in the vascular endothelial growth factor receptor (VEGFR) family, a receptor in the rearranged during transfection (RET) receptor family, a receptor in the Eph receptor family, a receptor that can induce cell differentiation (e.g., a Notch receptor), a cell adhesion molecule (CAM), an adhesion receptor such as integrin receptor, cadherin, selectin, and a receptor in the discoidin domain receptor (DDR) family, transforming growth factor beta receptor 1, and transforming growth factor beta receptor 2. In some embodiments, such a receptor is an immune cell receptor selected from a T cell receptor, a B cell receptor, a natural killer (NK) cell receptor, a macrophage receptor, a monocyte receptor, a neutrophil receptor, a dendritic cell receptor, a mast cell receptor, a basophil receptor, and an eosinophil receptor.

In certain embodiments, the cell is engineered to express a chimeric antigen receptor (CAR). According to some embodiments, the cell is engineered to express a recombinant TCR.

As described above, according to some embodiments, the cell may be engineered to express a CAR. The extracellular binding domain of the CAR may comprise a single chain antibody. The single-chain antibody may be a monoclonal single-chain antibody, a chimeric single-chain antibody, a humanized single-chain antibody, a fully human single-chain antibody, and/or the like. In one non-limiting example, the single chain antibody is a single chain variable fragment (scFv). In some embodiments, the extracellular binding domain of the CAR is a single-chain version (e.g., an scFv version) of an antibody approved by the United States Food and Drug Administration and/or the European Medicines Agency (EMA) for use as a therapeutic antibody, e.g., for inducing antibody-dependent cellular cytotoxicity (ADCC) of certain disease-associated cells in a patient, etc. Non-limiting examples of single-chain antibodies which may be employed when the protein of interest is a CAR include single-chain versions (e.g., scFv versions) of Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof.

When the methods of the present disclosure are performed on a cell engineered to express a recombinant receptor on its surface, the receptor may include one or more linker sequences between the various domains. A “variable region linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that includes the same light and heavy chain variable regions. A non-limiting example of a variable region linking sequence is a glycine-serine linker, such as a (G₄S)₃ linker as described above. In certain embodiments, a linker separates one or more heavy or light chain variable domains, hinge domains, transmembrane domains, co-stimulatory domains, and/or primary signaling domains. In particular embodiments, the receptor (e.g., CAR) includes one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids in length.

In some embodiments, when the methods of the present disclosure are performed on a cell engineered to express a recombinant receptor on its surface, the antigen binding domain of the receptor (e.g., CAR) is followed by one or more spacer domains that moves the antigen binding domain away from the cell surface (e.g., the surface of a T cell (e.g., a CD8+ or CD4+ T cell) expressing the receptor) to enable proper cell/cell contact, antigen binding and/or activation. The spacer domain (and any other spacer domains, linkers, and/or the like described herein) may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region. In some embodiments, the spacer domain includes the CH2 and/or CH3 of IgG1, IgG4, or IgD. Illustrative spacer domains suitable for use in the receptors (e.g., CARs) described herein include the hinge region derived from the extracellular regions of type 1 membrane proteins such as CD8a and CD4, which may be wild-type hinge regions from these molecules or variants thereof. In certain embodiments, the hinge domain includes a CD8a hinge region. According to some embodiments, the hinge is a PD-1 hinge or CD152 hinge. In certain embodiments, the hinge is an IgG4 hinge.

The “transmembrane domain” (Tm domain) is the portion of the receptor (e.g., CAR) that fuses the extracellular binding portion and intracellular signaling domain and anchors the receptor to the plasma membrane of the cell (e.g., T-cell, such as a Treg). The Tm domain may be derived either from a natural, synthetic, semi-synthetic, or recombinant source. In some embodiments, the Tm domain is derived from (e.g., includes at least the transmembrane region(s) or a functional portion thereof) of the alpha or beta chain of the T-cel receptor, CD35, CD3ζ, CD3γ, CD3δ, CD4, CD5, CD8α, CD9, CD16, CD22, CD27, CD28, CD33, CD37, CD45, CD64, CD80, CD86, CD134, CD137, CD152, CD154, or PD-1.

In one embodiment, a receptor (e.g., CAR) includes a Tm domain derived from CD28. In certain embodiments, a receptor includes a Tm domain derived from CD28 and a short oligo- or polypeptide linker, e.g., between 1, 2, 3, 4, 5, 6, 7, 8, 9, or 10 amino acids in length, that links the Tm domain and the intracellular signaling domain of the receptor. A glycine-serine linker may be employed as such a linker, for example.

The “intracellular signaling” domain of a receptor (e.g., a CAR) refers to the part of the receptor that participates in transducing the signal from binding to a target molecule/antigen into the interior of the cell to elicit cell function. Accordingly, the term “intracellular signaling domain” refers to the portion of a protein which transduces the signal and that directs the cell to perform a specialized function. To the extent that a truncated portion of an intracellular signaling domain is used, such truncated portion may be used in place of a full-length intracellular signaling domain as long as it transduces the signal. The term intracellular signaling domain is meant to include any truncated portion of an intracellular signaling domain sufficient for transducing signal.

Signals generated through the T cell receptor (TCR) alone are insufficient for full activation of the T cell, and a secondary or costimulatory signal is also required. Thus, T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. As such, a receptor (e.g., CAR) expressed by a cell genetically modified according to the methods of the present disclosure may include an intracellular signaling domain that includes one or more “costimulatory signaling domains” and a “primary signaling domain.”

Primary signaling domains regulate primary activation of the TCR complex either in a stimulatory manner, or in an inhibitory manner. Primary signaling domains that act in a stimulatory manner may contain signaling motifs which are known as immunoreceptor tyrosine-based activation motifs (or “ITAMs”). Non-limiting examples of ITAM-containing primary signaling domains suitable for use in a receptor of the present disclosure include those derived from FcRγ, FcRβ, CD3γ, CD3δ, CD3ε, CD3ζ, CD22, CD79α, CD79β, and CD66δ. In certain embodiments, a receptor includes a CD3ζ primary signaling domain and one or more costimulatory signaling domains. The intracellular primary signaling and costimulatory signaling domains are operably linked to the carboxyl terminus of the transmembrane domain.

In some embodiments, when the methods of the present disclosure are performed on a cell engineered to express a recombinant receptor on its surface, the receptor (e.g., CAR) includes one or more costimulatory signaling domains to enhance the efficacy and expansion of immune effector cells (e.g., T cells) expressing the receptor. As used herein, the term “costimulatory signaling domain” or “costimulatory domain” refers to an intracellular signaling domain of a costimulatory molecule or an active fragment thereof. Example costimulatory molecules suitable for use in receptors contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, KD2C, SLP76, TRIM, and ZAP70. In some embodiments, the receptor (e.g., CAR) includes one or more costimulatory signaling domains selected from the group consisting of 4-1BB (CD137), CD28, and CD134, and a CD34 primary signaling domain.

A receptor (e.g., CAR) expressed by a cel genetically modified according to the methods of the present disclosure may include any variety of suitable domains including but not limited to a leader sequence; hinge, spacer and/or linker domain(s); transmembrane domain(s); costimulatory domain(s); signaling domain(s) (e.g., CD3ζ domain(s)); ribosomal skip element(s); restriction enzyme sequence(s); reporter protein domains; and/or the like.

According to some embodiments, when the cell is engineered to express a receptor (e.g., a CAR) on its surface, the extracellular binding domain of the receptor specifically binds a tumor antigen expressed on the surface of a cancer cell. Non-limiting examples of tumor antigens to which the extracellular binding domain of the receptor may specifically bind include 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvIII, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside, glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), Tn antigen, trophoblast cell-surface antigen (TROP-2), Wilms' tumor 1 (WT1), and VEGF-A.

According to some embodiments, the cell may be engineered to express an antibody. The term “antibody” (also used interchangeably with “immunoglobulin”) encompasses antibodies of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the antigen, including, but not limited to single chain Fv (scFv), Fab, (Fab′)₂, (scFv′)₂, and diabodies; chimeric antibodies; monoclonal antibodies, humanized antibodies, human antibodies; and fusion proteins comprising an antigen-binding portion of an antibody and a non-antibody protein.

Immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 150 kDa or about 446 amino acids), similarly comprise a variable region (of about 116 amino acids) and one of the aforementioned heavy chain constant regions, e.g., gamma (of about 330 amino acids).

An immunoglobulin light or heavy chain variable region (V_{L} and V_{H}, respectively) is composed of a “framework” region (FR) interrupted by three hypervariable regions, also called “complementarity determining regions” or “CDRs”. The extent of the framework region and CDRs have been defined (see, E. Kabat et al., Sequences of proteins of immunological interest, 4th ed. U.S. Dept. Health and Human Services, Public Health Services, Bethesda, MD (1987); and Lefranc et al. IMGT, the international ImMunoGeneTics information System®. Nucl. Acids Res., 2005, 33, D593-D597)). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen. All CDRs and framework provided by the present disclosure are defined according to Kabat, supra, unless otherwise indicated.

An “antibody” thus encompasses a protein having one or more polypeptides that can be genetically encodable, e.g., by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. In some embodiments, an antibody of the present disclosure is an IgG antibody, e.g., an IgG1 antibody, such as a human IgG1 antibody. In some embodiments, the cell expresses an antibody that comprises a human Fc domain.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

### Cells and Compositions

Aspects of the present disclosure further include cells and compositions. For example, in certain aspects, provided is a population of cells genetically modified according to any of the methods of the present disclosure. Also provided are compositions comprising such cell populations.

In certain aspects, the compositions include any of the genetically modified cells of the present disclosure present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a solubalizing agent, a detergent (e.g., a non-ionic detergent such as Tween-20, etc.), a nuclease inhibitor, glycerol, a chelating agent, and the like may be present in such compositions.

The compositions generally include a therapeutically effective amount of the cells. By “therapeutically effective amount” is meant a number of cells sufficient to produce a desired result, e.g., an amount sufficient to effect beneficial or desired therapeutic (including preventative) results, such as a reduction in a symptom of a disease or disorder associated, e.g., with the target cell or a population thereof, as compared to a control. An effective amount can be administered in one or more administrations.

A “therapeutically effective amount” of such cells may vary according to factors such as the disease state, age, sex, and weight of the subject, and the ability of the cells to elicit a desired response in the subject. A therapeutically effective amount is also one in which any toxic or detrimental effects of the cells are outweighed by the therapeutically beneficial effects. The term “therapeutically effective amount” includes an amount that is effective to “treat” a subject (e.g., a patient). When a therapeutic amount is indicated, the precise amount of the compositions contemplated in particular embodiments, to be administered, can be determined by a physician in view of the specification and with consideration of individual differences in age, weight, tumor size, extent of infection or metastasis, and condition of the patient (subject). In certain aspects, a pharmaceutical composition of the present disclosure includes from 1×10⁶ to 5×10¹⁰ of the cells produced according to the methods of the present disclosure.

The cells of the present disclosure can be incorporated into a variety of formulations for therapeutic administration. More particularly, the cells of the present disclosure can be formulated for administration by combination with appropriate excipients, diluents and/or the like.

Formulations of the cells suitable for administration to a patient (e.g., suitable for human administration) are generally sterile and may further be free of detectable pyrogens or other contaminants contraindicated for administration to a patient according to a selected route of administration.

The cells may be formulated for parenteral (e.g., intravenous, intra-arterial, intraosseous, intramuscular, intracerebral, intracerebroventricular, intrathecal, subcutaneous, etc.) administration, or any other suitable route of administration.

An aqueous formulation of the cells may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included in the formulation to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

In some embodiments, a composition includes cells of the present disclosure, and one or more of the above-identified agents (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Methods of Administering Cell-Based Therapies

Aspects of the present disclosure further include methods of administering a cell-based therapy to a subject in need thereof, the methods comprising administering to the subject a therapeutically effective amount of cells genetically modified according to the methods of genetic modification of the present disclosure. For example, with the benefit of the present disclosure, it will be appreciated that altering (e.g., increasing) codon-anti-codon affinity via genetic modification of a cell according to the methods of the present disclosure may be employed to increase the proliferative capacity of cels (e.g., Immune cells such as T cells (e.g., CD8+ T cells, CD4+ T cells, Tregs, and/or the like), NK cells, etc.) and/or altered cell phenotype (e.g. memory, cytotoxic), where such increased proliferative capacity and/or alerted cell phenotype is advantageous in a variety of contexts including but not limited to cell-based therapies, e.g., CAR-T cell therapies, engineered T cell therapies (T cells that express engineered T cell receptors (TCRs)), and the like.

The cell or progeny thereof may be autologous/autogeneic (“self”) or non-autologous (“non-self,” e.g., allogeneic, syngeneic or xenogeneic). “Autologous” as used herein, refers to cells obtained from the subject to which the genetically modified cell(s) are later administered. “Allogeneic” as used herein refers to cells obtained from a donor other than the subject to which the genetically modified cell(s) are administered. In some embodiments, the cells (e.g., T cells) are cels obtained from a mammalian subject. In certain embodiments, the mammalian subject is a primate. In some embodiments, the cells are obtained from a human.

In a further aspect, provided are methods of administering a cell-based therapy to a subject, the methods comprising assessing cells obtained from a candidate donor for the presence or absence of a mutation in a coding region of a mitochondrial gene, where the mutation decreases translation of an mRNA encoded by the mitochondrial gene by decreasing the affinity of a codon-anti-codon interaction during translation of the mRNA as compared to the affinity of the codon-anti-codon interaction in the absence of the mutation. Such methods further comprise administering to the subject cells obtained from the candidate donor when the assessment determines the absence of the mutation in the cells obtained from the candidate donor, or administering to the subject cells obtained from a different donor when the assessment determines the presence of the mutation in the cells obtained from the candidate donor, where the mutation is not present in the cells obtained from the different donor. In certain embodiments, the mutation decreases the affinity of the codon-anti-codon interaction by converting the codon-anti-codon interaction from a non-wobble-dependent interaction to a wobble-dependent interaction. According to some embodiments, the protein encoded by the gene is an enzyme in the mitochondrial electron transport chain, e.g., MT-CO1 or any other enzyme in the mitochondrial electron transport chain described elsewhere herein. In certain embodiments, the candidate donor is the subject. According to some embodiments, the candidate donor is not the subject.

A variety of suitable approaches are available for assessing cells for the presence or absence of a mutation in a coding region of a mitochondrial gene. For example, an efficient method for sequencing the entire human mitochondrial genome directly from a biological sample obtained from a donor and its application to genetic testing is described in Yao et al. (2019) Scientific Reports 9:17411, the disclosure of which is incorporated herein by reference in its entirety for all purposes.

Any of the cell-based therapeutic methods of the present disclosure may be used to treat a variety of conditions in the subject. In certain embodiments, the subject has cancer. The methods may be employed for the treatment of a large variety of cancers. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be treated using the subject methods include, but are not limited to, carcinoma, lymphoma, blastoma, and sarcoma. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bile duct cancer, bladder cancer, hepatoma, breast cancer, colon cancer, endometrial or uterine carcinoma, salivary gland carcinoma, kidney cancer, prostate cancer, vulval cancer, thyroid cancer, hepatic carcinoma, various types of head and neck cancer, and the like. In certain embodiments, the individual has a cancer selected from a solid tumor, recurrent glioblastoma multiforme (GBM), non-small cell lung cancer, metastatic melanoma, melanoma, peritoneal cancer, epithelial ovarian cancer, glioblastoma multiforme (GBM), metastatic colorectal cancer, colorectal cancer, pancreatic ductal adenocarcinoma, squamous cel carcinoma, esophageal cancer, gastric cancer, neuroblastoma, fallopian tube cancer, bladder cancer, metastatic breast cancer, pancreatic cancer, soft tissue sarcoma, recurrent head and neck cancer squamous cell carcinoma, head and neck cancer, anaplastic astrocytoma, malignant pleural mesothelioma, squamous non-small cel lung cancer, rhabdomyosarcoma, metastatic renal cell carcinoma, basal cell carcinoma (basal cell epithelioma), and gliosarcoma. In certain aspects, the individual has a cancer selected from melanoma, Hodgkin lymphoma, renal cell carcinoma (RCC), bladder cancer, non-small cell lung cancer (NSCLC), and head and neck squamous cell carcinoma (HNSCC).

In certain embodiments, the cancer comprises a solid tumor. According to some embodiments, the solid tumor is a carcinoma or a sarcoma. When the solid tumor is a carcinoma, in certain embodiments, the carcinoma is a basal cell carcinoma, squamous cell carcinoma, renal cell carcinoma, ductal carcinoma in situ (DCIS), invasive ductal carcinoma, or adenocarcinoma.

By treatment is meant at least an amelioration of one or more symptoms associated with the condition of the subject, where amelioration is used in a broad sense to refer to at least a reduction in the magnitude of a parameter, e.g., symptom, associated with the condition being treated. As such, treatment also includes situations where the condition, or at least one or more symptoms associated therewith, are completely inhibited, e.g., prevented from happening, or stopped, e.g., terminated, such that the subject no longer suffers from the condition, or at least the symptoms that characterize the condition.

T cells may be obtained from a number of sources including, but not limited to, peripheral blood, peripheral blood mononuclear cells, bone marrow, lymph node tissue, cord blood, thymus tissue, tissue from a site of infection, ascites, pleural effusion, spleen tissue, and tumors. In certain embodiments, T cells can be obtained from a unit of blood collected from an individual using any number of known techniques such as sedimentation, e.g., FICOLL™ separation.

In some embodiments, an isolated or purified population of T cells is used. In some embodiments, after isolation of PBMC, both cytotoxic and helper T lymphocytes can be sorted into naive, memory, and effector T cell subpopulations either before or after activation, expansion, and/or genetic modification. Suitable approaches for such sorting are known and include, e.g., magnetic-activated cell sorting (MACS), where TN are CD45RA⁺ CD62L⁺ CD95⁻; TSCM are CD45RA⁺ CD62L⁺ CD95⁺; TCM are CD45RO⁺ CD62L⁺ CD95⁺; and TEM are CD45RO⁺ CD62L⁻ CD95⁺. An example approach for such sorting is described in Wang et al. (2016) Blood 127(24):2980-90.

A specific subpopulation of T cells expressing one or more of the following markers: CD3, CD4, CD8, CD28, CD45RA, CD45RO, CD62, CD127, and HLA-DR can be further isolated by positive or negative selection techniques. In one embodiment, a specific subpopulation of T cells, expressing one or more of the markers selected from the group consisting of CD62L, CCR7, CD28, CD27, CD122, CD127, CD197; or CD38 or CD62L, CD127, CD197, and CD38, is further isolated by positive or negative selection techniques. In various embodiments, the T cell compositions do not express or do not substantially express one or more of the following markers: CD57, CD244, CD 160, PD-1, CTLA4, TIM3, and LAG3.

In order to achieve sufficient therapeutic doses of T cell compositions, the T cells may be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes. In particular embodiments, T cells are activated and expanded for about 1 to 21 days, e.g., about 5 to 21 days. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to the genetic modification and/or introduction of a nucleic acid (e.g., expression construct) into the T cells.

In particular embodiments, T cells are activated and expanded for about 6 hours, about 12 hours, about 18 hours or about 24 hours prior to the genetic modification and/or introduction of a nucleic acid (e.g., expression construct) into the T cells. In certain aspects, T cells are activated concurrently with the genetic modification and/or introduction of a nucleic acid (e.g., expression construct) Into the T cells.

In some embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-γ, IL-4, IL-7, IL-21, GM-CSF, IL-10, IL-12, IL-15, TGFβ, and TNF-α or any other additives suitable for the growth of cells known to the skilled artisan. Further illustrative examples of cell culture media include, but are not limited to RPMI 1640, Clicks, AEVI-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cels.

The following examples are offered by way of illustration and not by way of limitation.

### EXPERIMENTAL

### Introduction

Synonymous variants in cellular DNA alter the sequence of genes but retain the amino acid sequence of the encoded protein, resulting in a typical interpretation of “silent” for the variant. However, synonymous variants in human mitochondrial DNA (mtDNA) have been linked with many human phenotypes via genome-wide association studies (GWAS) and have been recurrently observed in many human cancers, suggesting that these synonymous variants may play a functional role in disease pathophysiology.

Described herein is the identification of a mosaic synonymous mutation, m.7076A>G (MT-CO1:p:Gly391=), in peripheral blood mononuclear cells (PBMCs) from a healthy donor. While similar allelic heteroplasmy of this variant was observed in all hematopoietic lineages, a depletion of the m.7076G allele was observed specifically in the CD8⁺ TEM compartment, reminiscent of the selection of diverse pathogenic mtDNA variants. It is demonstrated herein that due to the limited diversity of the transfer RNA (tRNA) pool in mitochondria, this synonymous mutation requires translation via the super wobble effect, where a 5′ uracil in the anticodon can decode all four nucleotides in the 3′ codon. While capable of translation, the U-G wobble base pairing stalls mitochondrial ribosomes, thereby impeding CD8⁺ T differentiation. The principles of wobble-mediated translation elucidated from this mosaic variant enable a new ontogeny of synonymous mtDNA that broadly impact human genotypes and phenotypes. Specifically, provided herein is genetic evidence that germline and somatic synonymous mitochondrial variants undergo “codon optimization” In tumors, healthy tissues, and the germline across six different population-based cohorts (TCGA, IMPACT, PCAWG, GTEx, gnomAD, HelixMTdb). Comprehensive inter- and intra-species evolutionary analyses of the mitochondrial genome suggest that nucleotides in the wobble position experience accelerated evolutionary pressure between species, and human haplogroup-defining synonymous variation are enriched for variation consistent with preference for codon optimization. Based on population-level genotype associations from the UK BioBank (UKBB), synonymous variants that variably require wobble-dependent translation impact a variety of human traits and complex diseases. Altogether, delineated herein is a new ontology of functional synonymous variation altering codon syntax in the mitochondrial genome with broad relevance to human phenotypes, disease, and evolution.

Additional Examples and figures are provided in U.S. Provisional Patent Application No. 63/353,715, the disclosure of which is incorporated herein by reference in its entirety.

### Example 1—A Mosaic Synonymous mtDNA Variant that is Selected Against in the CD8⁺ T Cell Compartment

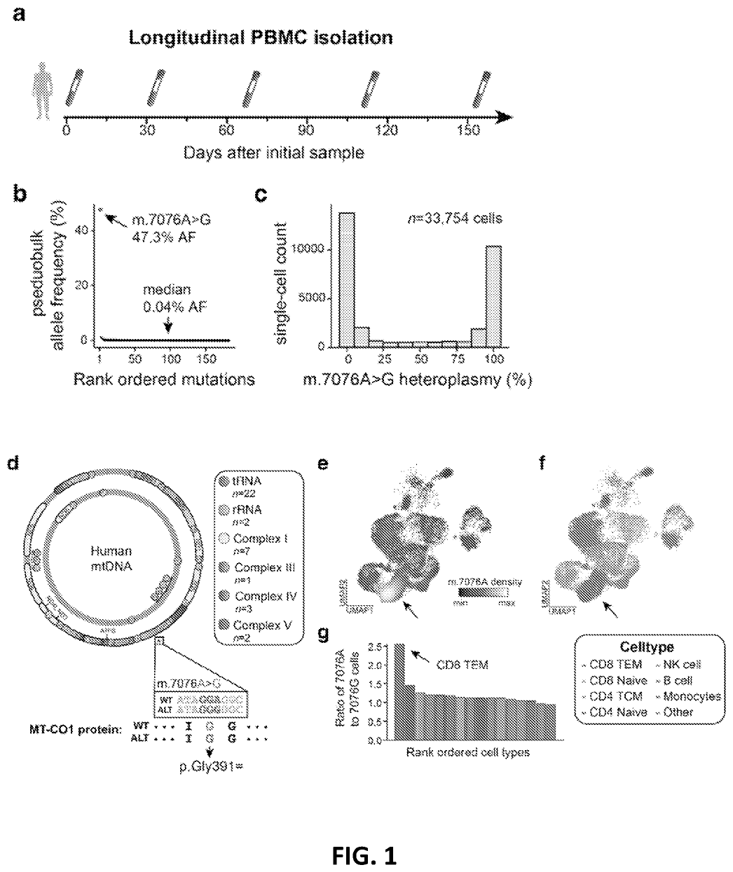

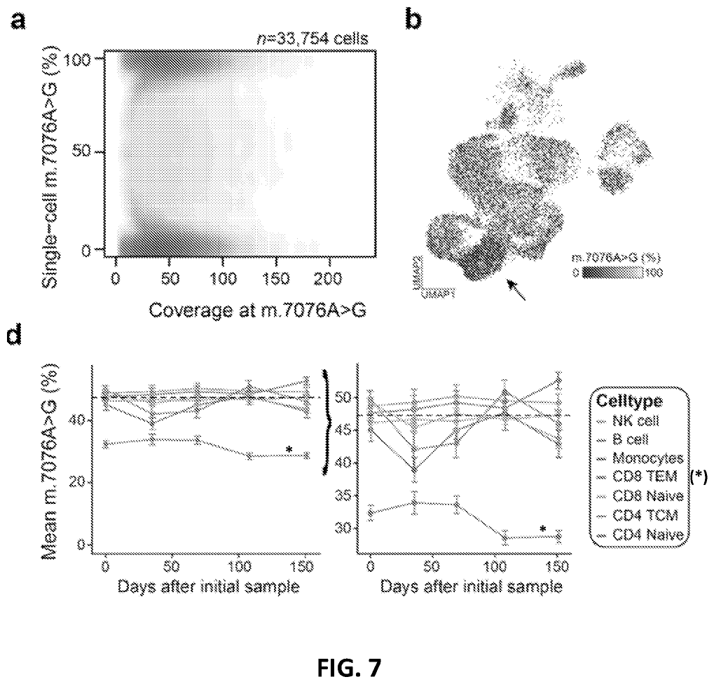

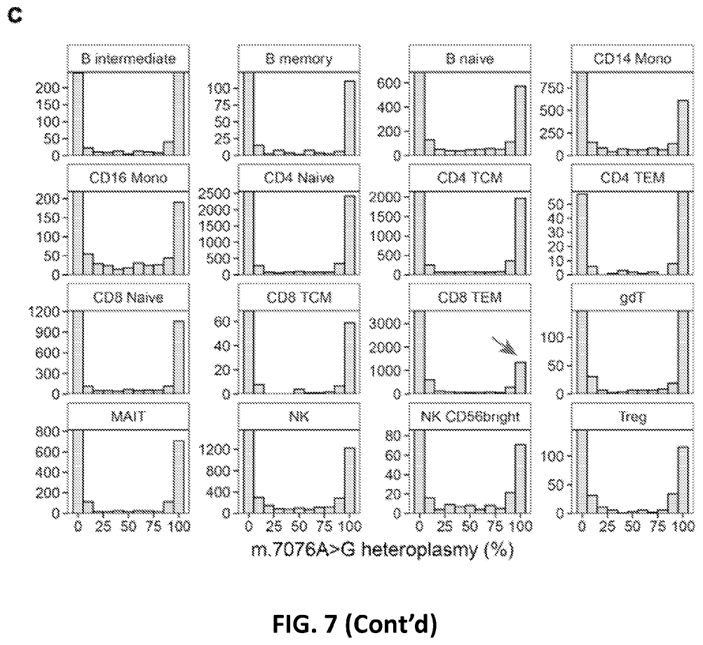

Over the course of longitudinal profiling of the clonal dynamics of a healthy donor, PBMCs were profiled with the mitochondrial scATAC-seq assay (mtscATAC-seq^{9,13}) to simultaneously yield cell state and mtDNA genotyping information from the same single cells (FIG. 1a). Consistent with prior observations from native hematopoiesis in healthy individuals⁹, identified were 183 somatic mtDNA variants with a median allele frequency of 0.04% in pseudobulk (FIG. 1b; Methods). Notably, observed was one variant, m.7076A>G, present at 47.3% heteroplasmy, suggesting this donor to be a chimera for this particular allele that likely arose during development. The m.7076A>G variant is a synonymous variant (p.Gly391=) in the mitochondrial cytochrome c oxidase subunit 1 (MT-CO1), a gene required for complex IV activity during oxidative phosphorylation (FIG. 1c-d). Strikingly, the majority of the 33,754 profiled cells had either 0% or 100% heteroplasmy (FIG. 1c, FIG. 7a), a pattern previously observed for a non-coding mutation m.16260C>T in an independent healthy donor¹¹. While m.7076A>G is present as a homoplasmic variant in 0.2%-0.9% of the population^{14,15}, the variant is not linked to a specific mitochondrial haplogroup. Still, approximately 1 in 15,000 individuals carries a highly heteroplasmic m.7076A>G allele (10-90% allele frequency) similar to this donor (Methods).

Leveraging the chromatin accessibility modality of the mtscATAC-seq dataset, a dictionary learning strategy was utilized to infer the cell states for each cell that was concomitantly genotyped (Methods). Strikingly, unlike in any other cel state, a marked depletion of cells homoplasmic for the mutant m.7076G allele was observed in a subset of CD8⁺ TEM (FIG. 1e,f, FIG. 7b-d). In other words, CD8⁺ TEMs had a marked increase of cells with the wildtype m.7076A allele, suggestive of lineage-specific selection pressure against cells with the mutant m.7076G allele (FIG. 1g). Notably, these findings mirror recent observations of purifying selection against pathogenic mtDNA mutations in CD8⁺ TEM cells in individuals with congenital mitochondriopathies, including patients with Mitochondrial Encephalopathy, Lactic Acidosis, and Stroke-like episode (MELAS)¹ and Pearson Syndrome², which is driven by the distinct demand for OXPHOS capacity during T cell proliferation and differentiation after activation^{16,17}.

### Example 2—Altered T-Cell State-Specific Gene Expression Due to m.7076A>G

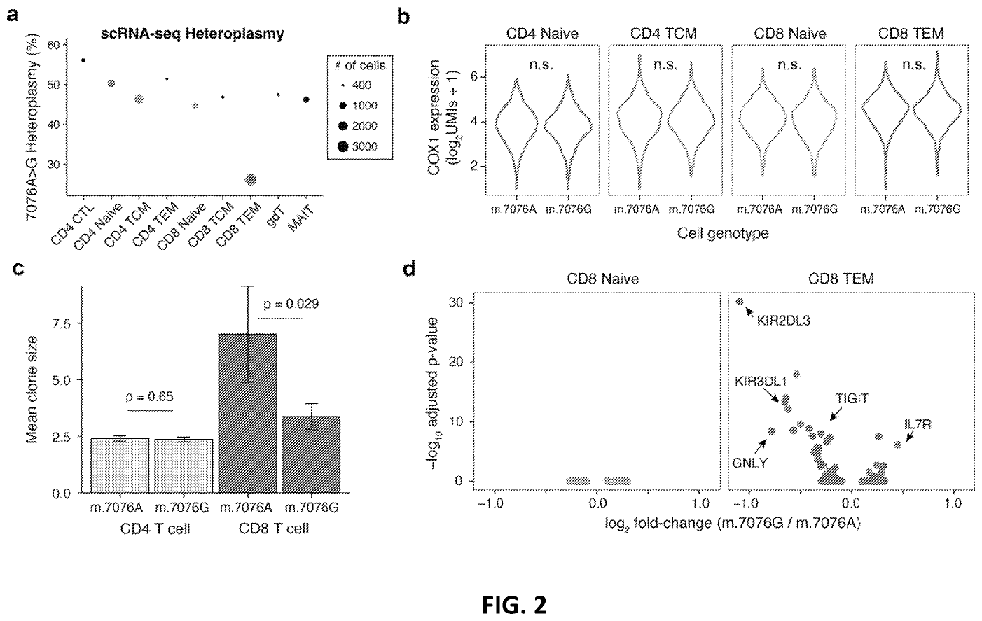

As synonymous variants after the sequence of DNA but retain the amino acid sequence of the encoded protein, these alleles do not tend to have a functional impact and are typically annotated as ‘silent’ mutations. However, differential effects of distinct tRNAs decoding synonymous codons and alterations in enzyme structure and function due to synonymous mutations have been reported¹⁸, including in human tumor progression^{19,20}. It was hypothesized that the synonymous variant m.7076A>G could incur a loss of function phenotype resulting in selective pressure during T cell differentiation analogous to known pathogenic mtDNA variants. To examine the possibility of this mutation impacting MT-CO1 mRNA expression, performed was single-cell RNA-seq (scRNA-seq) profiling of 17,337 T-cells derived from PBMCs derived from this donor. The specific depletion of the m.7076G allele in the CD8⁺ TEM compartment was verified, but not other T cell subsets (FIG. 2a). As a recent study attributed functional synonymous variation in yeast to differences in gene expression²¹, the abundance of the MT-CO1 transcript between cells with homoplasmy for either the wildtype or mutant allele was compared (Methods). Within any of these cell subsets, significant differences in expression were not observed, suggesting that the synonymous variant does not impact MT-CO1 stability or expression (FIG. 2b).

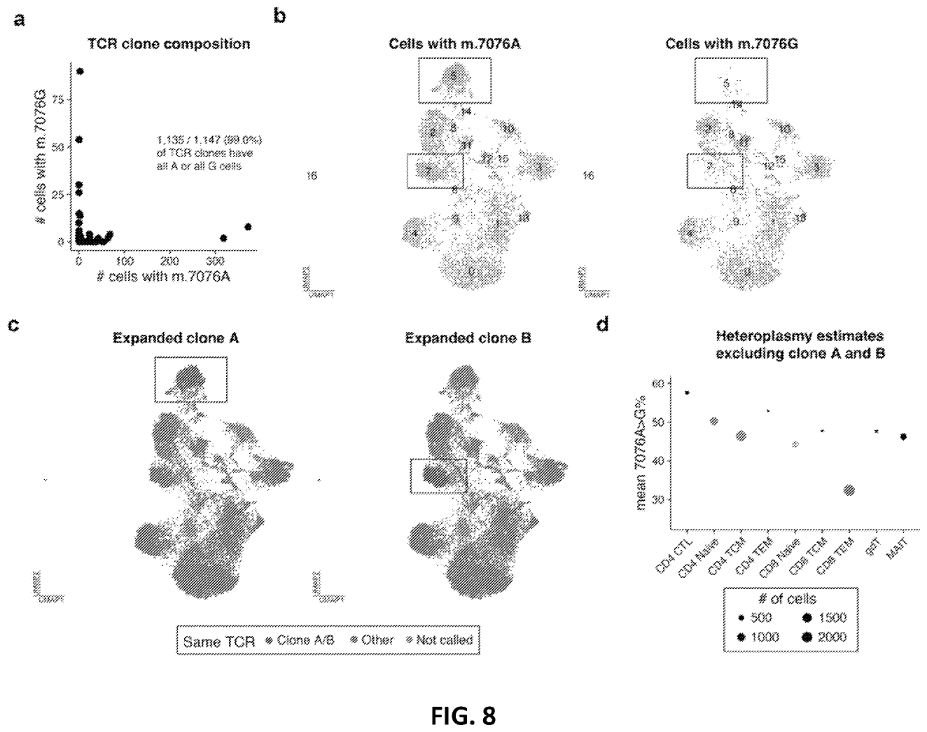

Utilizing concomitant single-cell T-cell receptor (TCR) sequencing, examined were clone sizes of CD4⁺ and CD8⁺ T cells with either m.7076 allele. As expected, TCR clones were effectively mutually exclusive for either m.7076 allele, confirming an early developmental origin of the mutant allele prior to TCR diversification (FIG. 8a). Observed was a diminished clone size in the CD8⁺ but not CD4⁺ T cell compartment in cells with the mutant m.7076G allele (FIG. 2c), suggesting reduced fitness or proliferative capacity of mutant CD8⁺ T cells to clonally expand. Further, differential gene expression analyses across T cell subsets was performed between wildtype and mutant m.7076 cells. Whereas all CD4⁺ subsets and CD8⁺ naive subsets had no differentially expressed genes between cells with distinct genotypes, 32 differentially expressed genes were observed between wildtype and mutant CD8⁺ TEM cels (FIG. 2d). While cytotoxic genes including GNLY, KIR2DL3, and KIR3DL1 were down-regulated, IL7R, a marker of naive T cells, was more highly expressed in cels with the mutant m.7076G allele. Additional unsupervised analyses revealed distinct cell states within the CD8⁺ TEM compartment, including highly clonal wildtype m.7076 allele populations expressing specific TCRs (FIG. 8b-c). However, these highly expanded clones cannot alone explain the observed depletion of the mutant allele in CD8⁺ TEM (FIG. 8d; Methods). Though the mutant m.7076A>G allele does not preclude the possibility of CD8⁺ TEM differentiation, it was concluded that mutant cells are at a competitive disadvantage to expand and acquire fully differentiated cytotoxic T cell-like phenotypes as evidenced by functional gene expression defects.

### Example 3—Refinement of Selected T Cell Phenotypes

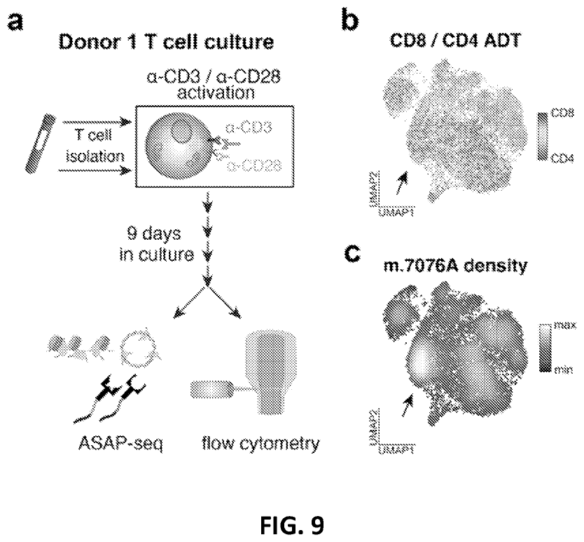

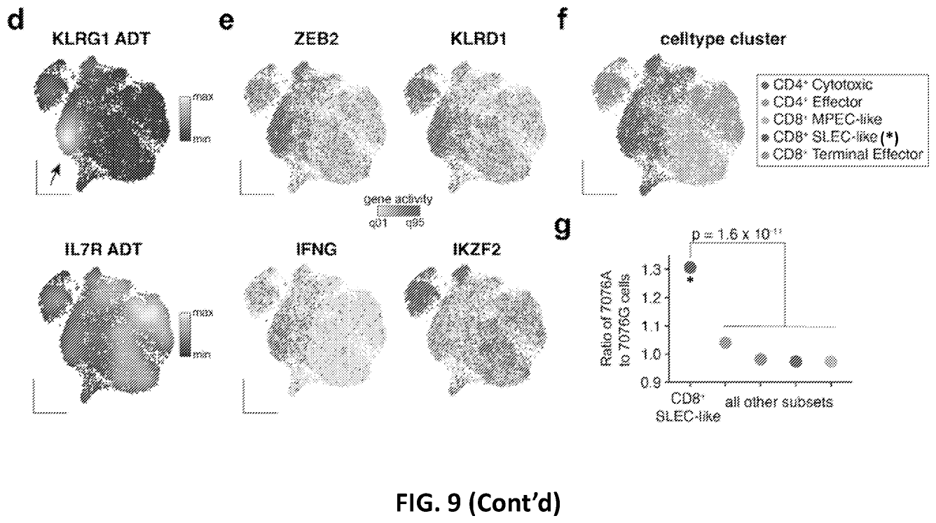

To further investigate the CD8⁺ T cell deficiencies incurred by this variant, PBMC-derived T cels were stimulated and cell phenotypes were examined using flow cytometry and ATAC with Selected Antigen Profiling via sequencing (ASAP-seq) that extends the mtscATAC-seq to co-quantify surface marker profiles via oligo-barcoded antibodies (FIG. 9a). Leveraging the multimodal measurements, observed again was a focalization of the wildtype m.7076A allele in a specific CD8⁺ T cell cluster (FIG. 9b,c). Interestingly, this cluster is characterized by high KLRG1 surface protein expression but depleted of IL7R protein, consistent with the transcriptomics data (FIG. 9d,e). Gene scores showed highly accessible chromatin in the ZEB2, IFN-γ, and KLRD1 loci, suggestive of a CD8⁺ short-lived effector cell (SLEC)-like population that is 1.3× enriched for cells with the wildtype m.7076A allele compared to other T cell subsets (FIG. 9f,g; p=1.6×10⁻¹¹; binomial test). Thus, the T-cell culture experiments confirmed mutant m.7076A>G cells to be impaired to attain specific CD8⁺ T cell states in vitro.

### Example 4—Altered Wobble-Dependent Translation of m.7076A>G

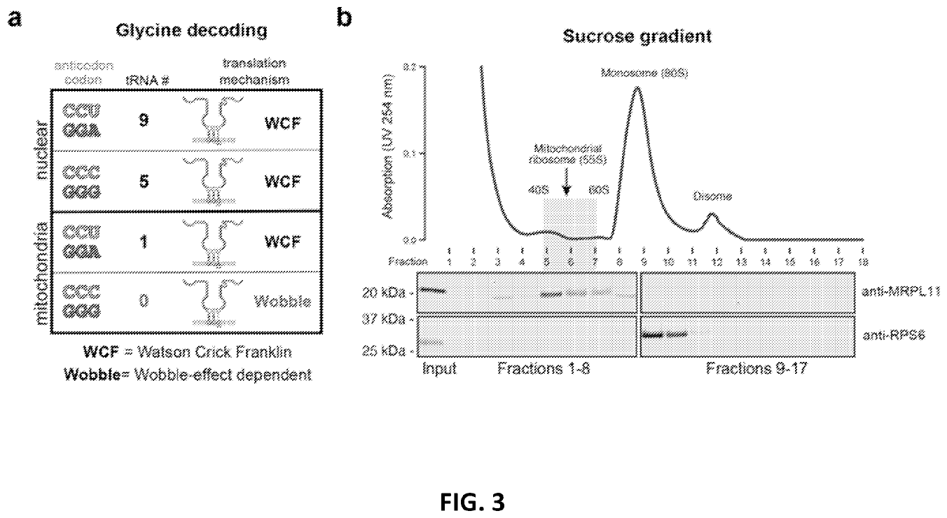

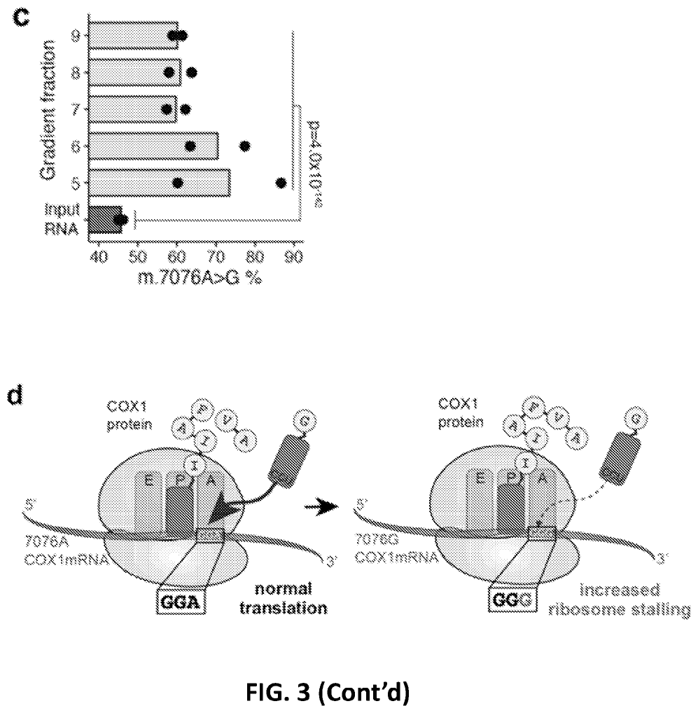

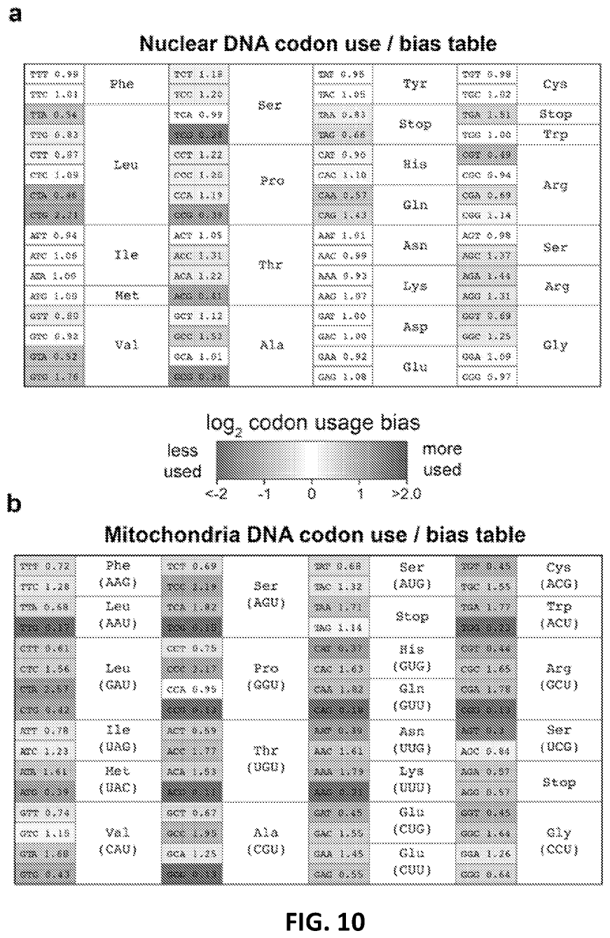

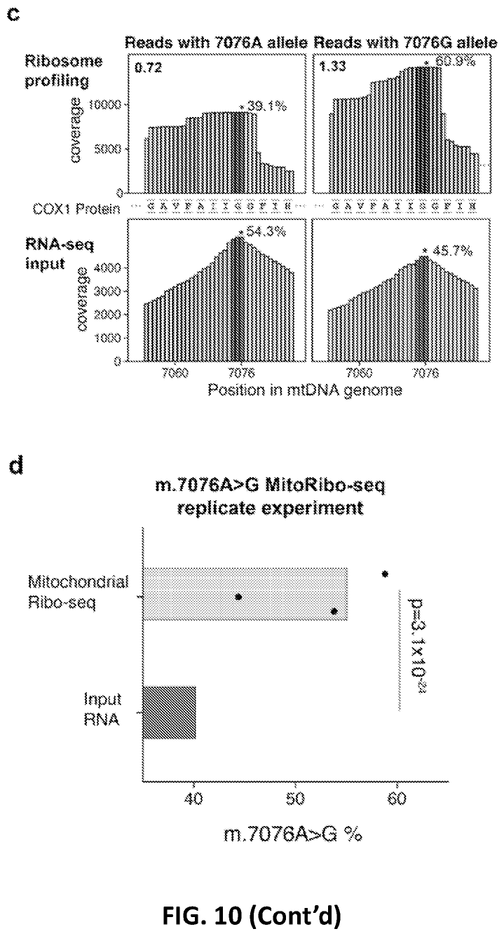

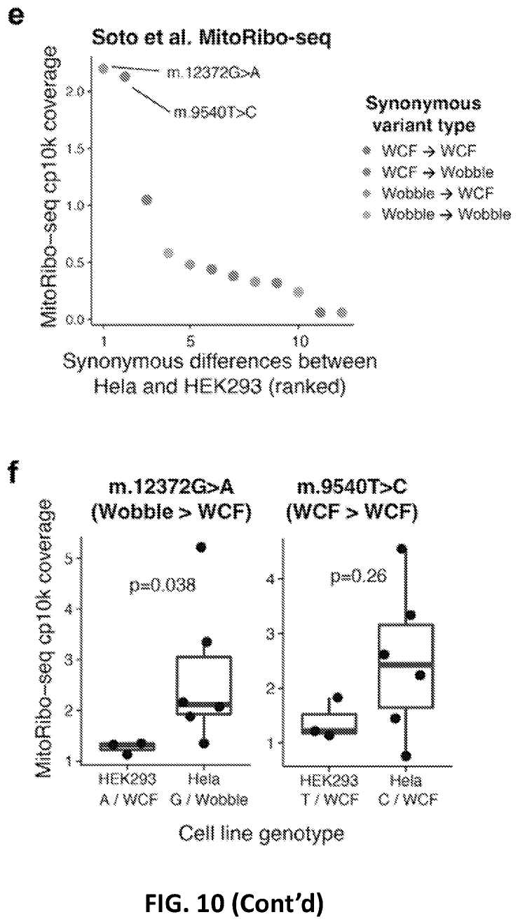

Given no alterations in MT-CO1 transcript abundance, a post-transcriptional mechanism was hypothesized to be responsible for the mutant functional effects. As synonymous codons may be decoded by independent tRNAs, revisited was the distinct pool of tRNAs in mitochondrial translation compared to nuclear-encoded tRNAs for cytoplasmic protein biosynthesis. Specifically, the nuclear genome redundantly encodes multiple tRNA loci for all possible codons, but only 22 tRNAs are ordinarily transcribed in the human mitochondrial genome. Thus, only a single glycine-specific tRNA (tRNA^{Gly}) is encoded in the mitochondrial genome to decode p.Gly391 via the wildtype GGA codon (m.7076A) or the mutant GGG codon (m.7076A>G; FIG. 3a). As such, decoding via the single mt-tRNA^{Gly} of the wildtype GGA allele occurs via canonical Watson-Crick-Franklin (WCF) base-pairing but requires wobble-dependent translation of the mutant GGG codon (FIG. 3a). Although the GGG codon is relatively depleted in the mitochondrial genome compared to the nuclear genome (FIG. 10a,b), this codon occurs at 34 positions within the 13 peptide-encoding open reading frames, suggesting the mitochondrial translational machinery to be generally capable of decoding this codon. Based on the cell state mapping experiments, it was hypothesized that the substantial requirement for oxidative phosphorylation during CD8⁺ T cell activation, differentiation, and proliferation may present a distinct metabolically vulnerable state toward CD8⁺ TEM cell state transition that is sensitive to even subtle changes in oxidative phosphorylation capacity.

To examine this possibility, PBMCs were stimulated in the presence of αCD3/αCD28 beads and IL-2, and mitochondrial translational efficiency was assessed (Methods). Fractions were isolated along a sucrose gradient to enrich mitochondrial ribosomes, and libraries were prepared for mitochondrial ribosome profiling by sequencing (MitoRiboSeq; FIG. 3b: Methods). Hypothesizing the altered codon syntax of the mutant m.7076A>G allele to impair translational decoding, the mutant G allele would be relatively enriched in the ribosome-protected fragments compared to the total mRNA input. Indeed, across all 10 fractions (5 per biological replicate), computed was a translation pause ratio, defined as the fraction of reads from ribosome profiling over the RNA-seq libraries, and the mutant m.7076A>G allele was observed to be enriched over the input material (FIG. 3c, FIG. 10c, mean 33% increase; p=4.0×10⁻¹⁴²), which was further replicated in an independent experiment (FIG. 10d, 3 additional replicates; mean 37% increase; p=3.1×10⁻²⁴). Specifically, the wildtype and mutant alleles were present at similar transcript levels (54.3% m.7076A vs. 45.7% m.7076G; ratio 1.19) compared to the mtscATAC-seq based genotyping results (47.3% m.7076G) further confirming MT-CO1 mRNAs abundance to be relatively unaltered. However, among ribosomal protected fragments, the mutant allele was substantially more abundant (39.1% wildtype m.7076A vs. 60.9% mutant m.7076A>G; ratio 0.64), strongly evident of translational staling around the mutant codon (FIG. 10c). Thus, the limited diversity of the tRNA pool prohibits efficient translation of the mutant m.7076A>G allele, otherwise requiring the super-wobble effect to effectively decode the mutant GGG codon. As a consequence, translation of MT-CO1 is stalled via a mechanism consistent with reports of super-wobble model systems²², thereby resulting in an effective partial LOF that becomes particularly restricting during the metabolically demanding process of CD8⁺ T cell differentiation.

### Example 5—A New Ontogeny of Synonymous mtDNA Variation

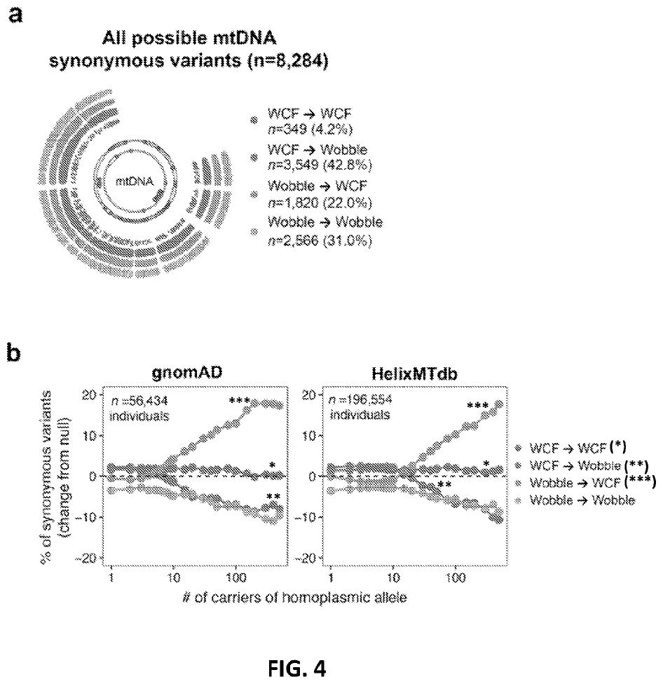

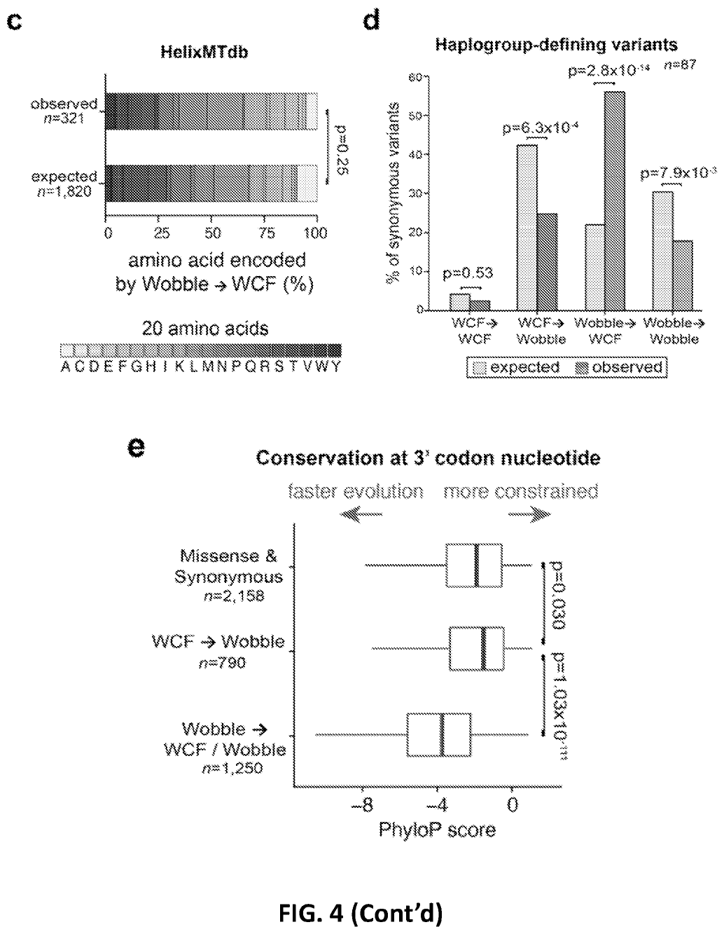

Given the overall restricted mitochondrial tRNA pool, the potential impact of synonymous mtDNA variation beyond the mutant m.7076A>G allele was evaluated considering all possible synonymous mutations across the mitochondrial genome, totaling 8,284 variants across 13 polypeptide-encoding genes (FIG. 4a). In the ontogeny, variants were annotated based on whether the reference and alternate alleles utilize canonical WCF or wobble base-pairing, leading to four possible classifications for synonymous variants: WCF→WCF, WCF→Wobble, Wobble→WCF and Wobble→Wobble (FIG. 4a; □ethods). From the annotation, it was observed that 48% of codons in the mitochondrial genome require wobble-dependent translation; however, potential functional effects of codon utilization in the human mitochondrial genome have apparently not been reported. To examine this annotation, public MitoRiboSeq data²³ from two cell lines (HEK293 and HeLa) were first examined and homoplasmic variants specific to each line were compared (Methods). For two additional mtDNA variants, sufficient coverage was observed to examine differences in MitoRiboSeq coverage, and confirmed was evidence of increased stalling due to a wobble-dependent base pairing but not a WCF pairing (FIG. 10e,f).

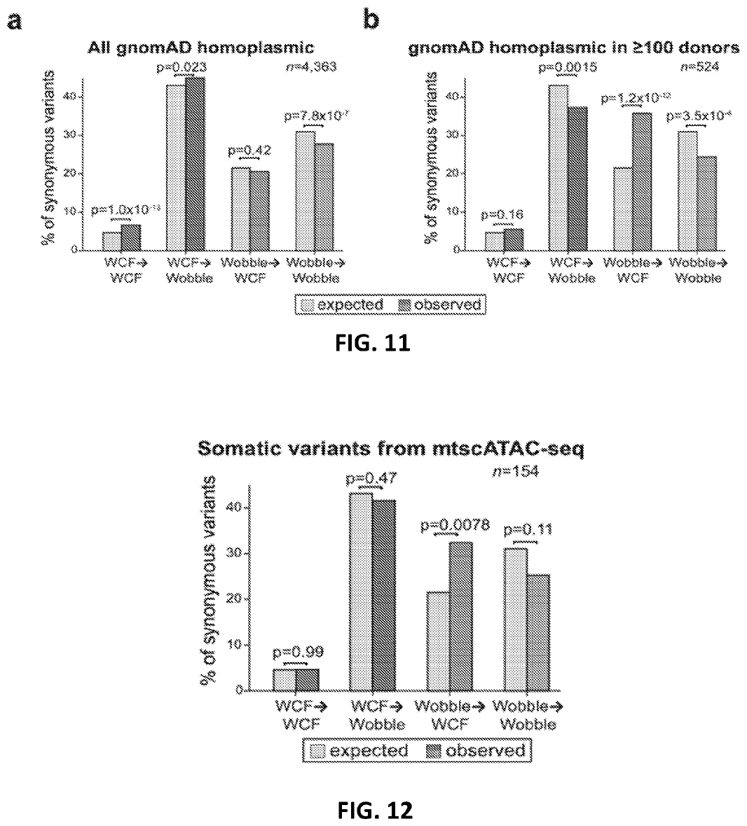

To assess the impact of this mitochondrial genome-wide annotation, queried were gnomAD²⁴ and HelixMTdb¹⁴, two comprehensive databases of germline variation in humans, where mtDNA variation has been called for 56,434 and 196,554 healthy individuals, respectively. While the simple occurrence of mtDNA variants (observed in ≥1 individual) mostly followed the expected proportions, the more common synonymous variants (present in multiple individuals) were strikingly enriched for variants that increased codon affinity (i.e., Wobble→WCF; “more optimal base-pairing”) and depleted for variants that reduced codon affinity (i.e., WCF→Wobble or Wobble→Wobble; “less optimal base-pairing”; FIG. 4b; FIG. 11a,b). This pattern of codon-optimizing variation increasing in the population was replicated between both gnomAD (p<2.2×10⁻¹⁶; Mann-Kendall Trend Test) and HelixMTdb (p=2.38×10⁻⁷; Mann-Kendall Trend Test). Wobble→WCF variants in at least 100 donors from HelixMTdb were observed in codons for all amino acids at proportions that did not deviate from the expected proportions (FIG. 4c; p=0.25; Chi-squared test), indicating that this selection for codon optimality impacted all amino acids. In total, the analyses demonstrate that variants impacting wobble-dependent translation are not constrained, consistent with recent work²⁵, but accumulate in a way that optimizes codon:anticodon affinity, indicative of positive selection at the population level.

### Example 6—mtDNA Wobble Codons Experience Evolutionary Selection

Investigated next was whether mitochondrial genomes displayed evolutionary evidence of codon selection. To examine this, both intra-species (FIG. 4d) and inter-species evolution was considered (FIG. 4e). Identified were 87 common synonymous variants linked to the phylogeny of human mitochondrial haplogroups^{26,27}, and their proportions in light of the new ontogeny were examined (Methods). Strikingly, observed was a >2.5 fold increase of Wobble→WCF variants (observed: 56.3%; expected: 22.0%; p=2.8×10⁻¹⁴), suggesting that variation linked to human mtDNA haplotypes for increased WCF pairing compared to the most recent common human ancestor is an ongoing evolutionary process. Considered next were inter-species annotations of conservation, requiring a per-position analysis rather than a per-variant analysis (Methods). Considering all three possible variant outcomes for a hypothetically mutated position, each of the wobble-position nucleotides in the mitochondrial genome were classified into three categories: positions that could variably lead to missense or synonymous variants (n=2,158), WCF reference alleles that become wobble in all scenarios (n=790), or wobble reference alleles that when mutated are either wobble or WCF (n=1,250). Using both a 20 and 100 species phyloP score²⁶, assessed was the evolutionary conservation of each codon class, confirming minimal constraint across any nucleotide consistent with prior results²⁵ (FIG. 4e). Notably, a significant decrease in phyloP score specifically for positions that require the wobble effect for translation in the reference genome was observed, suggesting that loci requiring the wobble effect are under accelerated evolution compared to already optimal WCF pairings (FIG. 4e; p=1.03×10⁻¹¹¹; Wilcoxon test). These results indicate that though human mtDNA possesses abundant codons that require wobble-dependent translation, the ongoing evolution of the human mtDNA genome is selecting for more optimal WCF-compatible base-pairing.

### Example 7—Complex Trait Associations

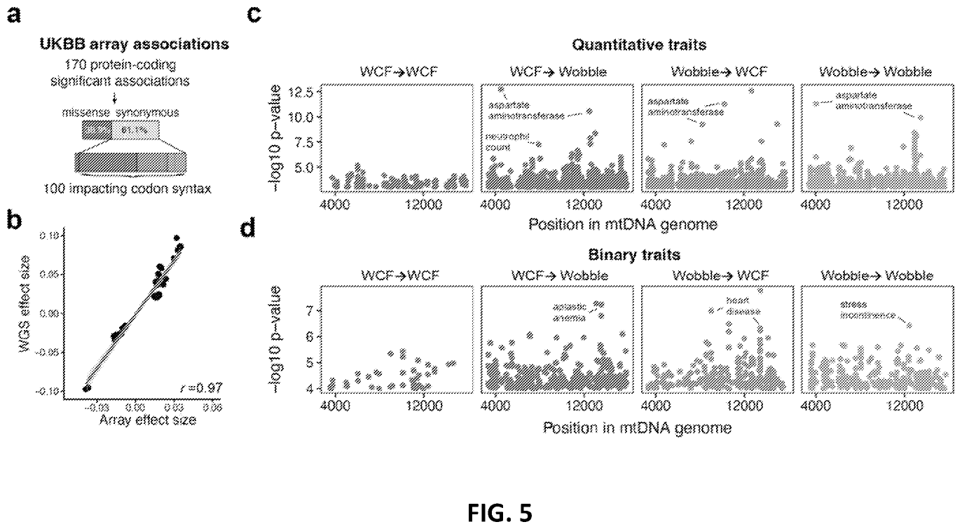

It was hypothesized that the classification of synonymous mtDNA variants would enable a new interpretation of results from genome-wide association studies. To assess this, reanalyzed were the association summary statistics from a recent study that examined the landscape of complex phenotypes from 488,377 individuals array-genotyped for 265 mtDNA SNVs in the UK Biobank while adjusting for nuclear/haplotype associations²⁹. Notably, most of the significantly associated SNPs in protein-coding genes were synonymous variants (104 of 170 or 61.1% of significant SNP-trait associations; FIG. 5a). 100 of the 104 synonymous variants altered codon syntax, more than a random sampling of variants (p-0.038; proportion test), including WCF→Wobble variants that confer risk for multiple sclerosis (m.13117A>G; MT-ND5; FDR=0.049) and type 2 diabetes (m.8655C>T; MT-ATP6; FDR=0.046). While prior studies of these synonymous variants suggested they tag a separate causal variant²⁹, these associations are interpreted to reflect the altered codon syntax impairing the effective translation and/or structure/function³⁰ of mitochondrial proteins, thereby impacting specific cell state function and differentiation, resulting in a heightened predisposition for a myriad of complex human traits.

As this mtDNA association analysis relied on variants present in a genotyping array, leveraged was the detection of the mitochondrial sequence information in whole-genome sequencing data to assess associations between complex traits and rare mtDNA variants (Methods). After verifying consistent effect estimates between the genotyping array and the WGS sequencing analyses (FIG. 5b), systematic association studies were performed between 2,786 commonly observed synonymous mtDNA variants with 1,656 quantitative phenotypes (FIG. 5c) and 18,688 binary phenotypes (FIG. 5d) from 171,673 UKBB participants (Methods). Using a permutation method to determine the statistical significance of the associations (Methods), identified were 492 variant-trait associations from the synonymous variants, only 12 of which were WCF→WCF. Further, WCF→WCF variants had a distinct distribution of test statistics compared to the other classes of synonymous variation altering codon affinity (p=9.6×10⁻⁷; Wilcoxon test), suggesting germline variants that alter mtDNA codon syntax may be broadly associated with complex human phenotypes.

The most significant associations were observed between several rare variants altering mitochondrial codon affinity and peripheral blood cell traits, including neutrophil count with m.7235C>A (Wobble→WCF; MT-CO1; p=2.7×10⁻⁸) as well as circulating metabolites and enzymes, including aspartate aminotransferase levels with m.4529A>T (WCF→Wobble; MT-ND2; p=1.7×10⁻¹³), m.4023T>C (Wobble→WCF; MT-ND5; p=5.1×10⁻¹²), and m.10238T>C (Wobble→Wobble; MT-ND1; p=5.2×10⁻¹²; FIG. 5c), which may be linked to mitochondrial metabolic activity in the liver³¹. Among disease phenotypes, observed were significant associations with multiple genitourinary diseases (m.10598A>G; p=3.3×10⁻⁷; MT-ND4L; Wobble→WCF) and aplastic anemia (m.13557A>G; p=5.9×10⁻⁸ MT-ND5; WCF→Wobble; FIG. 5d). For these highlighted variants, a functional effect in the mitochondria may be realized in a restricted cell state, analogous to the CD8⁺ T cell population implicated in the m.7076A>G mutation. In sum, the array- and sequencing-based analyses corroborate that the new annotation of wobble-dependent mtDNA variation broadly impacts complex traits via modulating genes encoded in the mitochondrial genome.

### Example 8—Codon Syntax is Somatically Optimized in Human Tumors and Tissues

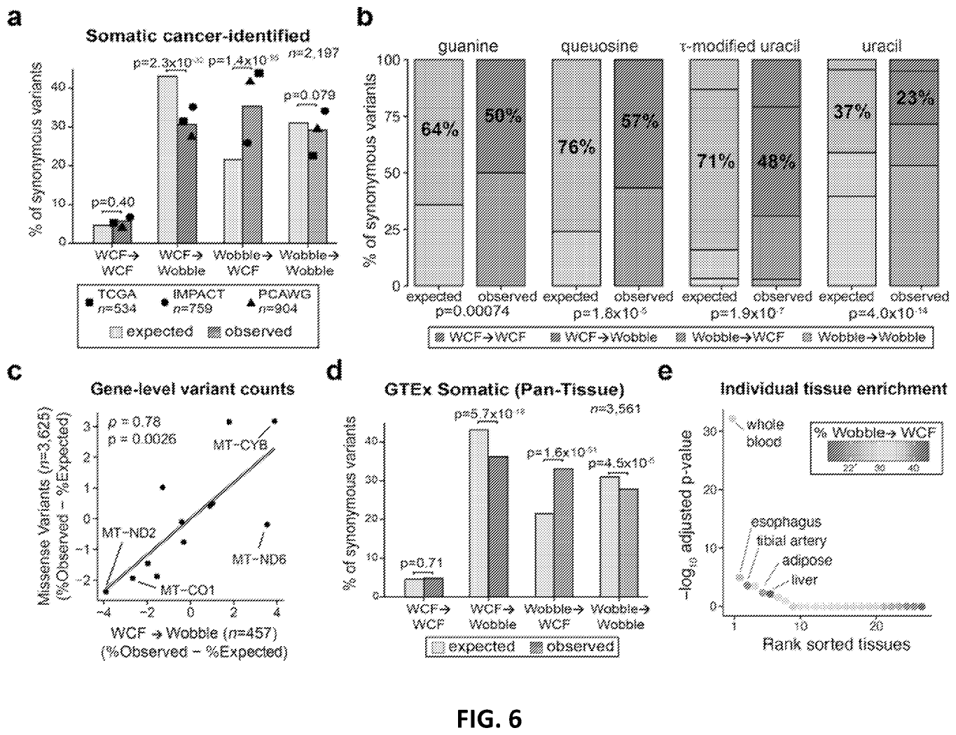

Systematic analyses of tumor sequencing data have suggested that translational control of the nuclear genome via codon usage can substantially modulate oncogenic processes^{20,32}. Thus, examined was a potential link between mtDNA synonymous mutation classes and somatic occurrence in tumors, and a total of 2,197 synonymous mtDNA mutations across three sequencing cohorts were analyzed (FIG. 6a). Notably, observed was a significant depletion of WCF→Wobble variants (“less optimal”; observed: 30.3%; expected: 42.8%; p=2.3×10⁻³²; proportion test) and a corresponding increase of Wobble→WCF (“more optimal”; observed: 35.9%; expected: 22.0%; p=1.4×10⁻⁵⁵; proportion test) across all cohorts. All possible synonymous mutations were stratified into four major classes based on the chemical structure of the wobble base to its paired tRNA (FIG. 6b; Methods). Many mitochondrial tRNAs are post-transcriptionally modified at the wobble position in the anticodon, leading to non-canonical nucleotides like queuosine, or uracil (super-wobble)³³. Irrespective of tRNA class, observed again was a consistent depletion of WCF→Wobble variants in the pan-cancer datasets (FIG. 6b), indicating this ontogeny of functional synonymous mtDNA likely impacts all amino acids and tRNA classes roughly equivalently.

As mutations in specific mitochondrial genes/complexes may modulate clinical outcome³⁴, the abundance of WCF→Wobble variants (“less optimal”) and missense variants at a gene level was examined compared to a null model (Methods). It was hypothesized that the per-gene mutational burden of these classes of variants might be correlated as they may result in partial loss of function phenotypes either due to protein-level changes or decreased translational efficiency. After quantifying 3,625 unique missense variants and 457 WCF→Wobble variants across the compendium of somatic tumor mtDNA mutations, a strong concordance between these two classes of genetic variants was observed; for example, MT-CYB was frequently mutated while MT-CO1 and MT-ND2 mutations were consistently depleted (FIG. 6c). This disparity is consistent with their differential roles in mitochondrial biology. Specifically, mild disruption of complex III (including MT-CYB) can be beneficial for tumor growth whereas mutations in genes in complex IV (e.g., MT-CO1) and complex I (e.g., MT-ND2) were unfavorable^{24,35}. These results reflect a broad model of somatic “codon optimization” in tumors, consistent with reports of enhanced OXPHOS activity to be favorable during oncogenesis, but noting that optimal translation rates can variably impact protein function¹⁸.

As this somatic codon optimization may be occurring in clonally expanded healthy tissues in addition to tumors, cross-tissue somatic mtDNA variation previously identified from the GTEx consortium¹³ were annotated. Observed was a global enrichment of Wobble→WCF variation across all tissues (p=1.6×10⁻⁵¹; proportion test; FIG. 6d). Examining individual tissues, observed were enrichments of codon-optimizing variation in seven tissues (adjusted p<0.05; proportion test), including whole blood, esophageal, tibial artery, adipose, and liver tissues (FIG. 6e), noting that this analysis is impacted by the overall prevalence of mutations (of which whole blood was most abundant). The enrichment of somatic Wobble→WCF variants in peripheral blood of somatic mtDNA variation was replicated with single-cell sequencing (FIG. 12a)². It was concluded that mutations in mitochondrial genomes experience selective pressure during tissue and tumor evolution, particularly in metabolically demanding cell states or cell state transitions, broadly leading to mitochondrial codon optimization.

### Example 9—Murine mtDNA Corroborates Functional Effect

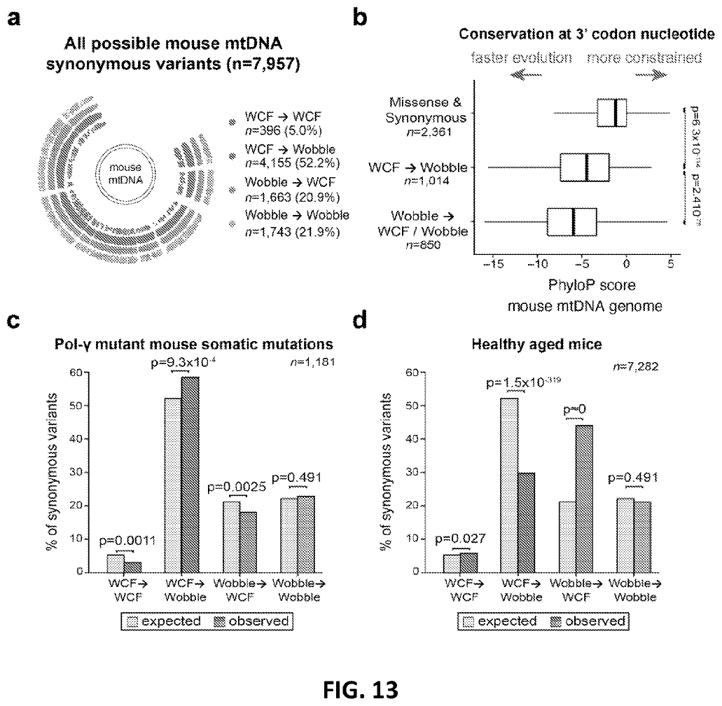

As eukaryotic mtDNA were derived from a common ancestor, it was hypothesized that the novel synonymous variant ontogeny should generalize to other species, including mice. Similarly annotated were each of the 7,957 possible synonymous variants in the mouse GRCm39 reference genome in light of wobble-mediated transition (FIG. 13a). This annotation broadly corroborated the inferences in humans, including the wobble nucleotides experiencing faster evolution than variants decreasing codon affinity or leading to missense mutations (FIG. 13b; Methods). Examined next was the somatic accumulation of synonymous mtDNA variation from pol-γ mutant mice³⁶ and in healthy aged mice³⁷ (FIG. 13c,d). Comparison of these mice revealed a striking disparity where pol-γ mutant mice had enrichment of WCF→Wobble variants (‘less optimal’; observed: 57.1% expected: 52.2%; p=0.00094; proportion test) whereas healthy aged mice had >2× enrichment of Wobble→WCF variants (‘more optimal’; observed: 43.8%; expected: 20.9%; p≈0; proportion test), a result consistent with the somatic occurrence in healthy human tissues via GTEx analyses (FIG. 6d). As the pol-γ mutant mice have deficient proof-reading capacities in the polymerase required for mitochondrial DNA replication, the increased accumulation of somatic mutations in these mice have been tied to distinct organismal phenotypes, including erythroid dysplasia and impaired lymphoesis³⁸. Thus, diminished codon affinity via synonymous variation may contribute to the functional effects of pol-γ mutant mice. These results culminate in a consistent model where mitochondrial genome syntax is frequently somatically optimized during tumorigenesis, germline variation, and clonal expansion in healthy tissues across multiple species.

### Methods

Mitochondrial Single-Cell ATAC-Seq (mtscATAC-Seq)

MtscATAC-seq libraries were generated using the 10× Chromium Controller and the Chromium Single Cell ATAC Library & Gel Bead Kit (#1000175) according to the manufacturer's instructions (CG000209-Rev G; CG000168-Rev B) as outlined below and previously described to increase mtDNA yield and genome coverage⁹. Briefly, 1.5 ml or 2 ml DNA LoBind tubes (Eppendorf) were used to wash cels in PBS and downstream processing steps. After washing cells were fixed in 1% formaldehyde (FA; ThermoFisher #28906) in PBS for 10 min at RT, quenched with glycine solution to a final concentration of 0.125 M before washing cells twice in PBS via centrifugation at 400 g, 5 min, 4° C. Cells were subsequently treated with lysis buffer (10 mM Tris-HCL pH 7.4, 10 mM NaCl, 3 mM MgCl₂, 0.1% NP40, 1% BSA) for 3 min for primary cells on ice, followed by adding 1 ml of chilled wash buffer and inversion (10 mM Tris-HCL pH 7.4, 10 mM NaCl, 3 mM MgCl₂, 1% BSA) before centrifugation at 500 g, 5 min, 4° C. The supernatant was discarded and cells were diluted in 1× Diluted Nuclei buffer (10× Genomics) before counting using Trypan Blue and a Countess II FL Automated Cel Counter. If large cell dumps were observed a 40 μm Flowmi cell strainer was used before processing cells according to the Chromium Single Cel ATAC Solution user guide with no additional modifications. Briefly, after tagmentation, the cells were loaded on a Chromium controller Single-Cell Instrument to generate single-cell Gel Bead-In-Emulsions (GEMs) followed by linear PCR as described in the protocol using a C1000 Touch Thermal cycler with 96-Deep Well Reaction Module (BioRad). After breaking the GEMs, the barcoded tagmented DNA was purified and further amplified to enable sample indexing and enrichment of scATAC-seq libraries. The final libraries were quantified using a Qubit dsDNA HS Assay kit (Invitrogen) and a High Sensitivity DNA chip run on a Bioanalyzer 2100 system (Agilent).

### Single-Cell RNA-Seq and TCR Profiling

Libraries for scRNA-seq were generated using the 10× Chromium Controller and the Chromium Single Cell 5′ Library Construction Kit and human B cell and T cell V(D)J enrichment kit according to the manufacturer's instructions. Briefly, the suspended cells were loaded on a Chromium controller Single-Cell Instrument to generate single-cell Gel Bead-In-Emulsions (GEMs) followed by reverse transcription and sample indexing using a C1000 Touch Thermal cycler with 96-Deep Wei Reaction Module (BioRad). After breaking the GEMs, the barcoded cDNA was purified and amplified, followed by fragmenting, A-tailing, and ligation with adaptors. Finally, PCR amplification was performed to enable sample indexing and enrichment of scRNA-seq libraries. For T-cel receptor sequencing, target enrichment from cDNA was conducted according to the manufacturer's instructions. The final libraries were quantified using a Qubit dsDNA HS Assay kit (Invitrogen) and a High Sensitivity DNA chip run on a Bioanalyzer 2100 system (Agilent). 10×scRNA-seq libraries were sequenced as recommended by the manufacturer (˜20,000 reads per cell) via a NovaSeq 6000 using an S4 flow cell.

ATAC with Selected Antigen Profiling by Sequencing (ASAP-Seq)

Cultured primary T cells were stained with a TSA-conjugated antibody panel (BioLegend ‘Universal’ Totalseq-A panel) that targets 154 distinct epitopes as previously described¹¹. Briefly, following sorting, cells were fixed in 1% formaldehyde and processed as described for the mtscATAC-seq workflow described above, with the modification that during the barcoding reaction, 0.5 μl of 1 μM bridge oligo A (BOA for TSA) was added to the barcoding mix. For GEM incubation the standard thermocycler conditions were used as described by 10× Genomics for scATAC-seq. Silane bead elution and SPRI cleanup steps were modified as described to generate the indexed protein tag library¹¹. The final libraries were quantified using a Qubit dsDNA HS Assay kit (Invitrogen) and a High Sensitivity DNA chip run on a Bioanalyzer 2100 system (Agilent). Libraries were amplified, sequenced, and preprocessed as previously described¹¹.

### Single-Cell ATAC-Seq Analyses

Raw sequencing data were demultiplexed using CellRanger-ATAC mkfastq. Demultiplexed sequencing reads for all libraries were aligned to the mtDNA blacklist modifieds hg38 reference genome using CellRanger-ATAC count v2.0. Mitochondrial DNA genotypes were determined using the mgatk workflow with default parameters⁹. Cell state analyses, including gene activity scores and surface protein visualization, were performed using the Seurat/Signac framework^{49,50}. For PBMC cell type annotations, granular cell type labels and UMAP coordinates were established by using the Seurat Dictionary Learning⁵¹ for cross-modality integration. Azimuth CITE-seq reference dataset labels⁵² with pubic 10× Genomics Multiome RNA- and ATAC-seq PBMC data were used as a cross-modality bridge. To assess whether additional genetic chimerism was present in cells marked with m.7076A>G, split was the full single-cell .bam file based on high-confidence cels with either the m.7076A or m.7076G allele and used FreeBayes⁵³ to genotype variants in the nuclear chromosomes using pseudobulk .bam files of the scATAC-seq profiles. After intersecting the two .vcf files from either the m.7076A or m.7076G variant calls, evidence of high-quality variants (QUAL>100) specific to either allele was not identified, noting that the variant calling was restricted to accessible chromatin regions.

### Single-Cell RNA-Seq Analyses

Raw sequencing data were demultiplexed using CellRanger mkfastq and aligned to the host reference genome using CellRanger v6.0 and TCR sequences were processed using the CellRanger vdj pipeline with default settings. Mitochondrial DNA genotypes were determined using the mgatk workflow with default parameters⁹ while accounting for UMIs using the -ub tag. T cell subset annotations were derived using the Azimuth CITE-seq reference dataset labels⁵² from healthy 10×PBMC data. Cells were assigned as m.7076A or m.7076G using a minimum coverage of 2 UMIs and homoplasmy for either allele from the transcriptomic data (mean coverage=3.2×/cell from 5′ scRNA-seq for m.7076). Expression levels of MT-CO1 were determined from the default normalization in Seurat and assessed for differences using a Wilcoxon test with the Benjamini-Hochberg adjustment for multiple testing.

### Mitochondrial Ribosome Profiling

Peripheral blood mononuclear cells were cultured in RPMI-1640 medium supplemented with 10% Fetal Bovine Serum (FBS), penicillin and streptomycin, and 10 ng/ml IL-2 (PeproTech) at 37° C., 5% CO₂. For in vitro expansion, cells were stimulated with Dynabeads Human T-Activator αCD3/αCD28 at a bead-to-cell ratio of 1:2 (11131D, Thermo Fisher Scientific). Cell counts were determined every 2-3 days, and maintained at a density of 1-2×10⁶ cells/ml. On day 7 after stimulation, cells were collected for mitochondrial ribosome profiling (MitoRiboSeq). Briefly, 0.1 mg/ml cycloheximide (CHX) and 0.1 mg/ml chloramphenicol (CAP) were directly added into the cell culture media followed by 5 min incubation at 37° C., 5% CO₂. 20×10⁸ cells were then centrifuged and washed with ice-cold PBS supplied with 0.1 mg/ml CHX and 0.1 mg/ml CAP. Dynabeads were magnetically removed, cells were pelleted, snap-frozen in liquid nitrogen, and stored at −80° C. until further processing.

The protocol for Mitochondrial ribosome enrichment was adapted from previous works. The flash-frozen cell pellet was resuspended in 1 mL of lysis buffer (20 mM Tris-HCl pH 8.0, 100 mM KCl, 10 mM MgCl2, 0.1 mg/mL chloramphenicol, 0.1 mg/mL cycloheximide, 1 mM DTT, 1% (vol/vol) Triton X-100, 0.1% (vol/vol) NP-40, 1× complete protease inhibitor cocktail, 20 U/mL RNasin, 20 U/mL DNase 1) and incubated for 10 min on ice. The lysate was homogenized by passing it 3 times through a 26 G needle. Cell debris was removed by centrifugation at 16,000 g for 10 min (4° C.) and 100 μL of clarified lysate was kept aside for preparation of RNA-seq libraries. Ribosome-protected fragments (RPFs) were generated by nuclease digestion. To this end, 3.75 U/μL of micrococcal nuclease (NEB) and 5 mM CaCl2 were added to the remaining cell lysate; the incubation was performed at room temperature with gentle shaking for 1 h. The reaction was quenched by adding 6 mM of EGTA. The digested lysate was loaded on top of a linear 5-45% sucrose gradient containing 20 mM Tris-HCl pH 8.0, 100 mM KCl, 10 mM MgCl2, 0.1 mg/mL chloramphenicol, 0.1 mg/mL cycloheximide and 1 mM DTT and centrifuged at 35,000 rpm, 4° C. for 2 hr using a Beckmann SW40 rotor. The gradient was then fractionated into 20 fractions of 0.57 mL using a Biocomp gradient station fractionator, which allowed the recording of a UV absorbance profile at 254 nm.

### Western Blot of Gradient Fractions

Western blot analysis was used to monitor the successful isolation of the 55S ribosome. Proteins were precipitated from 230 μL of each gradient fraction and 25 μL of input lysate by adding Trichloroacetic acid (TCA) to a final concentration of 20%. Samples were incubated on ice for 1-2 h and proteins were precipitated by centrifugation at 15,000 g at 4° C. The supernatant was removed and protein pellets were washed twice with 300 μL ice-cold acetone, followed by a 15 min centrifuging at 15,000 g and 4° C. Pellets were air-dried and resuspended in 30 fL 1× Lämmli-Buffer. Samples were separated on an 8 12% Bis-Tris SDS-PAGE gel and transferred to a nitrocellulose membrane using the iBlot/iBind system (Thermo Fisher Scientific). The presence of mitochondrial and cytoplasmic ribosomes in gradient fractions was estimated by incubating resulting membranes with anti-MRPL11 monoclonal antibody (D68F2, Cell Signaling) and anti-RPS6 rabbit monoclonal antibody (Cell Signaling).

### RPF Isolation and Library Generation

Ribosome-protected fragments were isolated using Phenol/Chloroform extraction. To that end, 300 μl of the corresponding gradient fractions were mixed with equal amounts of Phenol:Chloroform:IAA (25:24:1, pH 6.6), transferred to a PhaseLock tube (VWR International), and centrifuged at 15,000 g for 5 min. A second clean-up step was performed using the isolated aqueous phase and adding equal amounts of Chloroform. After centrifugation, the aqueous phase was isolated and purified using the RNA Clean & Concentrator kit (Zymo Research) by following the manufacturer's instructions for small RNAs. Subsequent library preparation was performed as previously described⁵⁵ with the following modification: ribosomal RNA depletion was skipped for RPF libraries due to low starting material. For the preparation of matched RNA-seq libraries, total RNA was extracted from 40 μL of clarified input lysate using the Direct-zol RNA MiniPrep Kit (Zymo Research). Subsequently, rRNA was depleted using rboPOOLs (siTOOLs Biotech) and following the manufacturer's instructions. Subsequently, RNA-seq libraries were generated as described previously⁵⁵.

### MitoRibo-Seq Analyses

Raw .fastq files were trimmed for adapter sequences using cutadapt and subsequently aligned with bwa mem using default parameters as previously described⁵⁵. Summary statistics of coverage and m.7076A>G heteroplasmy were determined using the mgatk workflow with default parameters as well as the -kd flag to retain duplicate fragments with the same start and end coordinates. Pileup coverages of the locus spit by the m.7076 allele were determined using a custom python script using pysam library.

To examine other variants aside from m.7076A>G that may participate in wobble-mediated translational stalling, utilized was high-quality HEK293 (n=3) and Hela (n=6) MitoRibo-seq profiles from a recent manuscript²³ at GEO accession GSE173283. Raw RNA-seq reads were processed and variants distinct between the two cell lines were inferred using mgatk⁹. Identified were two synonymous variants that were homoplasmic but with distinct alleles in the two cell lines that had mean counts per 10,000 reads (cp10 k) greater than 2 in the MitoRibo-seq data.

### Annotation of Synonymous Variation in the Mitochondrial Genome

The revised Cambridge reference sequence (used in GRCh37, GRCh38, and hg38) was used as the basis for the ontogeny of somatic variation. Utilized were all possible mtDNA variations and protein-coding annotations as previously described¹⁰. Using this landscape of 8,284 synonymous mutations, annotated was whether the codon in the reference or the alternate allele would create a canonical (i.e., Watson-Crick-Franklin, WCF) base-pairing between the codon and anticodon or would require wobble-dependent base-pairing for translation. Once these annotations were established, the null model of the abundance of each variant class (FIG. 4) was derived from the empirical distribution of these variant classes in the full mitochondrial genome. To compare population-level enrichment, the gnomAD v3.1 variant call set via the MT .vcf file available on the download page²⁴ was used and the HelixMTdb database available online was parsed. For the analysis of somatic variation in cancer samples (FIG. 6), utilized were three pan-cancer datasets of somatic mtDNA variation derived from tumors that were previously reported³⁴. For the overall analysis of somatic cancer mutations (colored bars), the variant lists were concatenated, which effectively provided a weighted average proportional to the number of mutations called per study. GTEx analyses of somatic variants were determined using a previous catalog that filtered somatic variants occurring in more than one BioSample to reduce potential biases from variant calling in transcriptomics data¹³.

To further separate synonymous mtDNA variants based on tRNA class (FIG. 6b), categorized were each of the 22 human mt-tRNAs based on one of five categories using the chemical RNA structures previously annotated³³. These classes were determined based on the chemical structure of the base in the wobble position anticodon (position 34 in the tRNA structure). Four of the five classes and chemical structures are shown in FIG. 6b with the fifth being the f₅C34 which is restricted to the methionine codon, which were excluded from the analysis as it was only one tRNA and two total codons. Otherwise, each variant was annotated with the tRNA responsible for translation based on the canonical mtRNA translation table³³.

### Evolutionary and Cross-Species Analyses

DNA variants from the mitochondrial genome used to define haplogroups were determined from the PhyloTree Build 17 annotation as distributed by HaploGrep2^{26,27}. Filtered variants required a phylogenetic recurrence of 10, leading to 289 variants, including 48 missense and 87 synonymous variants. To calculate the inter-species evolution of the mitochondrial genome, utilized was the phyloP annotation per nucleotide (rather than per variant and this metric is not available from phyloP), requiring the annotation of wobble-position nucleotides in the mitochondrial genome to be assigned to one of three categories (FIG. 4b). The associations shown in FIG. 4b are for a 20-species phyloP calculation but were consistent using a 100-species estimation as well. Both phyloP annotations were downloaded from UCSC Genome Browser for the GRCh38 genome annotation. Similar analyses for the murine mm10 genome, including a 60-species phyloP, are shown in FIG. 13. Somatic variants from the pol-γ mutant mice³⁶ and in healthy aging mice³⁷ were downloaded from the supplemental tables from prior publications.

### Complex Human Trait Analysis

### Cohort and Phenotypes

The UK Biobank is a prospective study of approximately 500,000 participants 40-69 years of age at recruitment. Participants were recruited in the UK between 2006 and 2010 and are continuously followed. The average age at recruitment for sequenced individuals was 56.5 years. Participant data include health records that are periodically updated by the UKB, self-reported survey information, linkage to death and cancer registries, collection of urine and blood biomarkers, imaging data, accelerometer data, genetic data, and various other phenotypic endpoints. All study participants provided informed consent.

The UKB phenotype data was harmonized as previously described. Briefly, two main phenotypic categories were studied: binary and quantitative traits taken from the December 2021 data release (UKB application 26041). Phenotypic data was parsed using a previously described R package, PEACOK (github.com/astrazeneca-cgr-publications/PEACOCK). In addition, as previously described, relevant ICD-10 and ICD-9 codes were grouped into clinically meaningful “Union” phenotypes as previously described⁵⁶. For all binary phenotypes, controls were matched by sex when the percentage of female cases was significantly different (Fisher's exact two-sided P<0.05) from the percentage of available female controls. In total, associations with 18,688 binary phenotypes and 1,656 quantitative phenotypes were assessed.

### Whole-Genome Sequencing

Whole-genome sequencing (WGS) data of the UKB participants were generated by deCODE Genetics and the Wellcome Trust Sanger Institute as part of a public-private partnership involving AstraZeneca, Amgen, GlaxoSmithKline, Johnson & Johnson, Wellcome Trust Sanger, UK Research and Innovation, and the UKB. These individuals were pseudorandomly selected from the set of UKB participants. The WGS sequencing methods have been previously described. Briefly, genomic DNA underwent paired-end sequencing on Illumina NovaSeq6000 instruments with a read length of 2×151 and an average coverage of 32.5×. Conversion of sequencing data in BCL format to FASTQ format and the assignments of paired-end sequence reads to samples were based on 10-base barcodes, using bcl2fastq v2.19.0. Initial quality control was performed by deCODE and Wellcome Sanger, which included sex discordance, contamination, unresolved duplicate sequences, and discordance with microarray genotyping data checks. A total of 199,949 genomes passed these quality control measures.

UK Biobank genomes were processed at AstraZeneca using the provided CRAM format files. A custom-built Amazon Web Services (AWS) cloud compute platform running Illumina DRAGEN Bio-IT Platform Germline Pipeline v3.7.8 was used to align the reads to the GRCh38 genome reference and to call small variants, including on the mitochondrial genome where a continuous allele frequency model is used; a single alternate allele is considered as a candidate variant and an allele fraction is estimated for emitted variants. All PASS variants emitted had a confidence score (LOD) above the default of 6.3. Mitochondrial SNVs and indels were annotated using SnpEff v4.3⁵⁷ against Ensembl Build 38.92⁵⁸ with the use of the vertebrate mitochondrial amino acid code configured using ‘hg38.M.codonTable:Vertebrate_Mitochondrial’ and ‘hg38.MT.codonTable:Vertebrate_Mitochondrial’.

From an initial cohort of 199,949 genomes, 120 were removed where the genome showed kinship <0.49 (<98% Identity) to the exome from the same participant, where available; 8 that could not be linked to a participant; 0 with ≥4% contamination estimated by VerifyBamID⁵⁹; 5 where self-reported gender did not match karyotypic sex inferred by X:Y coverage ratio across CCDS release 22 bases; 0 where <94.5% of CCDS r22 bases had ≥10-fold coverage; 100 in the top 0.05% of CCDS r22 coverage (>67.2×); and 4,915 to obtain a set with all pairwise kinship estimates ≤0.1769 based on KING.

Europeans are the most well-represented genetic ancestry in the UKB. The participants with European genetic ancestry were identified based on Peddy⁶⁰ v0.4.2 Pr(EUR)>0.95. Performed then was finer-scale ancestry pruning of these individuals, retaining those within four standard deviations from the mean across the first four principal components, resulting in a final set of 183,116 not closely related genomes of European ancestry for analysis.

### Variant-Level Association Tests

Considering all 8,284 possible synonymous variants, variant-level association tests were performed for the 2,786 synonymous variants by requiring the alternate allele to be observed in at least six individuals of European ancestry. A two-sided Fisher's exact test was used for binary traits and linear regression was used for quantitative traits (correcting for age and sex). For binary traits, only included were phenotypes with at least 30 cases. The alternate allele read fraction was dichotomized into a binary genotype indicator variable, with fractions ≥0.90 coded as 1 and fractions ≤0.10 (including the absence of a variant call) coded as zero. Intermediate fractions were set to missing genotypes. Variants were required to have a mapping quality score (MQ)≥40 and DRAGEN variant status PASS, i.e. not filtered with lod_fstar or base_quality.

Performed was an n-of-1 permutation test, as previously described in the exome-based phenome-wide association study, to determine an appropriate p-value threshold. Briefly, the case-control (or quantitative measurement) labels were shuffled once for every phenotype while maintaining the participant-genotype structure. At a p-value threshold of 1×10⁻⁶, a total of 8/52,064,768 binary and 1/4,613,616 associations in the permuted analysis were significant, suggesting a negligible false positive rate for the thresholds reported in the main text, and thus used as a conservative threshold for analysis and interpretation.

### REFERENCES

1. Walker, M. A. et al. Purifying Selection against Pathogenic Mitochondrial DNA in Human T Cells. N. Engl. J. Med. 383, 1556-1563 (2020).
2. Lareau, C. A. et al. Single-cell multi-omics reveals dynamics of purifying selection of pathogenic mitochondrial DNA across human immune cells. Preprint at doi.org/10.1101/2022.11.20.517242.
3. Khajuria, R. K. et al. Ribosome Levels Selectively Regulate Translation and Lineage Commitment in Human Hematopoiesis. Cell 173, 90-103.e19 (2018).
4. Stewart, J. B. & Chinnery, P. F. The dynamics of mitochondrial DNA heteroplasmy: Implications for human health and disease. Nat. Rev. Genet. 16, 530-542 (2015).
5. Yazar, S. et al. Single-cell eQTL mapping identifies cell type-specific genetic control of autoimmune disease. Science 376, eabf3041 (2022).
6. Nathan, A. et al. Single-cell eQTL models reveal dynamic T cell state dependence of disease loci. Nature 606, 120-128 (2022).
7. Nam, A. S. et al. Single-cell multi-omics of human clonal hematopoiesis reveals that DNMT3A R882 mutations perturb early progenitor states through selective hypomethylation. Nat. Genet. 54, 1514-1526 (2022).
8. Nam, A. S. et al. Somatic mutations and cel identity linked by Genotyping of Transcriptomes. Nature 571, 355-360 (2019).
9. Lareau, C. A. et al. Massively parallel single-cell mitochondrial DNA genotyping and chromatin profiling. Nat. Biotechnol. (2020) doi:10.1038/s41587-020-0645-6.
10. Miller, T. E. et al. Mitochondrial variant enrichment from high-throughput single-cell RNA sequencing resolves clonal populations. Nat. Biotechnol. 40, 1030-1034 (2022).
11. Mimitou, E. P. et al. Scalable, multimodal profiling of chromatin accessibility, gene expression and protein levels in single cels. Nat. Biotechnol. (2021) doi:10.1038/s41587-021-00927-2.
12. Fiskin, E. et al. Single-cell profiling of proteins and chromatin accessibility using PHAGE-ATAC. Nat. Biotechnol. (2021) doi:10.1038/s41587-021-01065-5.
13. Ludwig, L. S. et al. Lineage Tracing in Humans Enabled by Mitochondrial Mutations and Single-Cell Genomics. Cell 176, 1325-1339.e22 (2019).
14. Bolze, A. et al. A catalog of homoplasmic and heteroplasmic mitochondrial DNA variants in humans. Preprint at doi.org/10.1101/798264.
15. Karczewski, K. J., Francioli, L. C. & MacArthur, D. G. The mutational constraint spectrum quantified from variation in 141,456 humans. Yearbook of Paediatric Endocrinology Preprint at doi.org/10.1530/ey.17.14.3 (2020).
16. Jones, N. et al. Metabolic Adaptation of Human CD4+ and CD8+ T-Cells to T-Cell Receptor-Mediated Stimulation. Front. Immunol. 8, 1516 (2017).
17. van der Windt, G. J. W. et al. Mitochondrial respiratory capacity is a critical regulator of CD8+ T cell memory development. Immunity 36, 68-78 (2012).
18. Jiang, Y. et al. How synonymous mutations after enzyme structure and function over long timescales. Nat. Chem. (2022) doi:10.1038/s41557-022-01091-z.
19. Earnest-Noble, L. B. et al. Two isoleucyl tRNAs that decode synonymous codons divergently regulate breast cancer metastatic growth by controlling translation of proliferation-regulating genes. Nat Cancer 3, 1484-1497 (2022).
20. Goodarzi, H. et al. Modulated Expression of Specific tRNAs Drives Gene Expression and Cancer Progression. Cell 165, 1416-1427 (2016).
21. Shen, X., Song, S., Li, C. & Zhang, J. Synonymous mutations in representative yeast genes are mostly strongly non-neutral. Nature 606, 725-731 (2022).
22. Rogalski, M., Karcher, D. & Bock, R. Superwobbling facilitates translation with reduced tRNA sets. Nat. Struct. Mol. Biol. 15, 192-198 (2008).
23. Soto, I. et al. Balanced mitochondrial and cytosolic translatomes underlie the biogenesis of human respiratory complexes. Preprint at doi.org/10.1101/2021.05.31.446345.
24. Laricchia, K. M. et al. Mitochondrial DNA variation across 56,434 individuals in gnomAD. Genome Res. (2022) doi:10.1101/gr.276013.121.
25. Lake, N. J. et al. Quantifying constraint in human mitochondrial DNA. bioRxiv 2022.12.16.520778 (2022) doi:10.1101/2022.12.16.520778.
26. Weissensteiner, H. et al. HaploGrep 2: mitochondrial haplogroup classification in the era of high-throughput sequencing. Nucleic Acids Res. 44, W58-63 (2016).
27. van Oven, M. PhyloTree Build 17: Growing the human mitochondrial DNA tree. Forensic Science International: Genetics Supplement Series 5, e392-e394 (2015).
28. Pollard, K. S., Hubisz, M. J., Rosenbloom, K. R. & Siepel, A. Detection of nonneutral substitution rates on mammalian phylogenies. Genome Res. 20, 110-121 (2010).
29. Yonova-Doing, E. et al. An atlas of mitochondrial DNA genotype-phenotype associations in the UK Biobank. Nat. Genet. 53, 982-993 (2021).
30. Jiang, Y. et al. How synonymous mutations alter enzyme structure and function over long time scales. bioRxiv 2021.08.18.456802 (2022) doi:10.1101/2021.08.18.456802.
31. Sookolan, S. & Pirola, C. J. Alanine and aspartate aminotransferase and glutamine-cycling pathway: their roles in pathogenesis of metabolic syndrome. World J. Gastroenterol. 18, 3775-3781(2012).
32. Gillen, S. L., Waldron, J. A. & Bushell, M. Codon optimality in cancer. Oncogene 40, 6309-6320 (2021).
33. Suzuki, T. et al. Complete chemical structures of human mitochondrial tRNAs. Nat. Commun. 11, 4269 (2020).
34. Gorelick, A. N. et al. Respiratory complex and tissue lineage drive recurrent mutations in tumour mtDNA. Nat Metab (2021) doi:10.1038/s42255-021-00378-8.
35. Martinez-Reyes, I. et al. Mitochondrial ubiquinol oxidation is necessary for tumour growth. Nature 585, 288-292 (2020).
36. Maclaine, K. D., Stebbings, K. A., Llano, D. A. & Havird, J. C. The mtDNA mutation spectrum in the PoIG mutator mouse reveals germline and somatic selection. BMC Genom Data 22, 52 (2021).
37. Sanchez-Contreras, M. et al. Multi-tissue landscape of somatic mtDNA mutations indicates tissue specific accumulation and removal in aging. bioRxiv 2022.08.30.505884 (2022) dol:10.1101/2022.08.30.505884.
38. Chen, M. L. et al. Erythroid dysplasia, megaloblastic anemia, and impaired lymphopoiesis arising from mitochondrial dysfunction. Blood 114, 4045-4053 (2009).
39. Lee-Six, H. et al. The landscape of somatic mutation in normal colorectal epithelial cells. Nature 574, 532-537 (2019).
40. Yoshida, K. et al. Tobacco smoking and somatic mutations in human bronchial epithelium. Nature 578, 266-272 (2020).
41. Lee-Six, H. et al. Population dynamics of normal human blood inferred from somatic mutations. Nature 561, 473-478 (2018).
42. Brunner, S. F. et al. Somatic mutations and clonal dynamics in healthy and cirrhotic human liver. Nature 574, 538-542 (2019).
43. Crimi, M. et al. Mitochondrial-DNA nucleotides G4298A and T10010C as pathogenic mutations: the confirmation in two new cases. Mitochondrion 3, 279-283 (2004).
44. Dhindsa, R. S. et al. A minimal role for synonymous variation in human disease. bioRxiv 2022.07.13.499964 (2022) doi:10.1101/2022.07.13.499964.
45. Shen, X., Song, S., Li, C. & Zhang, J. On the fitness effects and disease relevance of synonymous mutations. bioRxiv 2022.08.22.504687 (2022) doi:10.1101/2022.08.22.504687.
46. Kruglyak, L. et al. No evidence that synonymous mutations in yeast genes are mostly deleterious. Preprint at doi.org/10.1101/2022.07.14.500130.
47. Sharp, P. M., Bailes, E., Grocock, R. J., Peden, J. F. & Sockett, R. E. Variation in the strength of selected codon usage bias among bacteria. Nucleic Acids Res. 33, 1141-1153 (2005).
48. Boguszewska, K., Szewczuk, M., Kaźmierczak-Barańska, J. & Karwowski, B. T. The Similarities between Human Mitochondria and Bacteria in the Context of Structure, Genome, and Base Excision Repair System. Molecules 25, (2020).
49. Stuart, T., Srivastava, A., Madad, S., Lareau, C. A. & Satija, R. Single-cell chromatin state analysis with Signac. Nat. Methods 18, 1333-1341 (2021).
50. Stuart, T. et al. Comprehensive Integration of Single-Cell Data. Cell 177, 1888-1902.e21 (2019).
51. Hao, Y. et al. Dictionary learning for integrative, multimodal, and scalable single-cell analysis. bioRxiv 2022.02.24.481684 (2022) doi:10.1101/2022.02.24.481684.
52. Hao, Y. et al. Integrated analysis of multimodal single-cell data. Cell 184, 3573-3587.e29 (2021).
53. Garrison, E. & Marth, G. Haplotype-based variant detection from short-read sequencing. arXiv [q-bio.GN](2012).
54. Li, S. H.-J., Nofal, M., Parsons, L. R., Rabinowitz, J. D. & Gitai, Z. Monitoring mammalian mitochondrial translation with MitoRiboSeq. Nat. Protoc. 16, 2802-2825 (2021).
55. Basak, A. et al. Control of human hemoglobin switching by LIN28B-mediated regulation of BCL11A translation. Nat. Genet. 52, 138-145 (2020).
56. Wang, Q. et al. Rare variant contribution to human disease in 281,104 UK Biobank exomes. Nature 597, 527-532 (2021).
57. Cingolani, P. et al. A program for annotating and predicting the effects of single nucleotide polymorphisms, SnpEff: SNPs in the genome of Drosophila melanogaster strain w1118; iso-2; iso-3. Fly 6, 80-92 (2012).
58. Cunningham, F. et al. Ensembl 2022. Nucleic Acids Res. 50, D988-D995 (2022).
59. Jun, G. et al. Detecting and estimating contamination of human DNA samples in sequencing and array-based genotype data. Am. J. Hum. Genet. 91, 839-848 (2012).
60. Pedersen, B. S. & Quinlan, A. R. Who's Who? Detecting and Resolving Sample Anomalies in Human DNA Sequencing Studies with Peddy. Am. J. Hum. Genet. 100, 406-413 (2017).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A method of genetically modifying a cell, the method comprising:
modifying a coding region of a mitochondrial gene of the cell, wherein:
(i) the modification results in increased translation of a messenger RNA (mRNA) encoded by the mitochondrial gene by increasing the affinity of a codon-anti-codon interaction during translation of the mRNA as compared to the affinity of the codon-anti-codon interaction prior to the modifying; or
(ii) the modification results in decreased translation of an mRNA encoded by the mitochondrial gene by decreasing the affinity of a codon-anti-codon interaction during translation of the mRNA as compared to the affinity of the codon-anti-codon interaction prior to the modifying.

2. The method according to claim 1, wherein the modification results in increased translation of the mRNA.

3. The method according to claim 2, wherein the modifying converts the codon-anti-codon interaction from a wobble-dependent interaction to a non-wobble-dependent interaction.

4. The method according to claim 2 or claim 3, wherein the protein encoded by the gene is an enzyme in the mitochondrial electron transport chain.

5. The method according to claim 4, wherein the enzyme is mitochondrially encoded cytochrome C oxidase I (MT-CO1).

6. The method according to claim 1, wherein the modification results in decreased translation of the mRNA, and wherein the modifying converts the codon-anti-codon interaction from a non-wobble-dependent interaction to a wobble-dependent interaction.

7. A method of genetically modifying a cell, wherein translation of a messenger RNA (mRNA) encoded by a mitochondrial gene of the cell is wobble-dependent, the method comprising:
introducing into mitochondria of the cell an expression construct from which a transfer RNA (tRNA) is transcribed, wherein the anti-codon of the tRNA is selected such that translation of the mRNA encoded by the mitochondrial gene is no longer wobble-dependent.

8. A method of genetically modifying a cell, the method comprising:
introducing into mitochondria of the cell:
a first nucleic acid encoding a tRNA; and
a second nucleic acid encoding a protein,
wherein translation of the protein encoded by the second nucleic acid is wobble-dependent in the absence of the tRNA and non-wobble-dependent in the presence of the tRNA.

9. The method according to claim 8, wherein transcription of the tRNA encoded by the first nucleic acid is inducible.

10. The method according to claim 9, wherein transcription of the tRNA is induced upon activation of a signaling pathway of the cell.

11. The method according to any one of claims 8 to 10, wherein the first nucleic acid and the second nucleic acid are the same nucleic acid.

12. The method according to any one of claims 8 to 10, wherein the first nucleic acid and the second nucleic acid are separate nucleic acids.

13. The method according to any one of claims 1 to 12, wherein the cell is an immune cel.

14. The method according to claim 13, wherein cell is a T cell.

15. The method according to claim 14, wherein the T cell is a CD8⁺ T cell.

16. The method according to claim 14, wherein the T cell is a CD4⁺ T cell.

17. The method according to claim 14, wherein the T cell is a regulatory T cell (Treg).

18. The method according to claim 13, wherein cell is a natural killer (NK) cell.

19. The method according to any one of claims 13 to 18, wherein prior to, subsequent to, or concurrently with the modifying, the cell is engineered to express a recombinant receptor on its surface.

20. The method according to claim 19, wherein the recombinant receptor is a chimeric antigen receptor (CAR).

21. The method according to any one of claims 14 to 17, wherein the T cell is engineered to express a recombinant T cell receptor (TCR) on its surface.

22. The method according to any one of claims 1 to 21, further comprising administering the cell or progeny thereof to a subject in need thereof.

23. The method according to claim 22, wherein the cell or progeny thereof are autologous to the subject.

24. The method according to claim 22, wherein the cell or progeny thereof are allogeneic to the subject.

25. A population of cels genetically modified according to the method of any one of claims 1 to 21.

26. A composition comprising the population of cells of claim 25.

27. The composition of claim 26, wherein the composition is formulated for administration to a subject.

28. A method of administering a cell-based therapy to a subject, the method comprising:
assessing cells obtained from a candidate donor for the presence or absence of a mutation in a coding region of a mitochondrial gene, wherein the mutation decreases translation of an mRNA encoded by the mitochondrial gene by decreasing the affinity of a codon-anti-codon interaction during translation of the mRNA as compared to the affinity of the codon-anti-codon interaction in the absence of the mutation; and
administering to the subject cells obtained from the candidate donor when the assessment determines the absence of the mutation in the cells obtained from the candidate donor, or
administering to the subject cells obtained from a different donor when the assessment determines the presence of the mutation in the cells obtained from the candidate donor, wherein the mutation is not present in the cells obtained from the different donor.

29. The method according to claim 28, wherein the mutation decreases the affinity of the codon-anti-codon interaction by converting the codon-anti-codon interaction from a non-wobble-dependent interaction to a wobble-dependent interaction.

30. The method according to claim 28 or claim 29, wherein the protein encoded by the gene is an enzyme in the mitochondrial electron transport chain.

31. The method according to claim 30, wherein the enzyme is MT-CO1.

32. The method according to any one of claims 28 to 31, wherein the cells are immune cells.

33. The method according to claim 32, wherein the immune cells are T cells.

34. The method according to claim 33, wherein the T cells are CD8⁺ T cells.

35. The method according to claim 33, wherein the T cells are CD4⁺ T cells.

36. The method according to claim 33, wherein the T cells are Tregs.

37. The method according to claim 32, wherein the immune cells are NK cels.

38. The method according to any one of claims 32 to 37, wherein the cells administered to the subject are engineered to express a recombinant receptor on their surface.

39. The method according to claim 38, wherein the recombinant receptor is a chimeric antigen receptor (CAR).

40. The method according to any one of claims 33 to 36, wherein the T cells are engineered to express a recombinant T cell receptor (TCR) on their surface.

41. The method according to any one of claims 28 to 40, wherein the candidate donor is the subject.

42. The method according to any one of claims 28 to 40, wherein the candidate donor is not the subject.
